(19) United States
(12) Patent Application Publication
(10) Pub. No.: US 2025/0195896 A1
(43) Pub. Date: Jun. 19, 2025

(54) CATHETER AND METHOD FOR DETECTING DYSSYNERGY RESULTING FROM DYSSYNCHRONY

(71) Applicant: Pacertool AS, Oslo (NO)

(72) Inventor: Hans Henrik ODLAND, Oslo (NO)

(21) Appl. No.: 18/844,903

(22) PCT Filed: Mar. 7, 2023
(86) PCT No.: PCT/EP2023/055799

§ 371 Date: Sep. 6, 2024

(30) Foreign Application Priority Data
Mar. 8, 2022 (GB) 2203204.9

(51) Int. Cl. A61N 1/365; A61N 1/05

(52) U.S. Cl. CPC A61N 1/36585; A61N 1/056

(57) ABSTRACT

A catheter and method for detecting dyssynergy resulting from dyssynchrony There is provided a catheter for assessing cardiac function, the catheter comprising an elongate shaft extending from a proximal end to a distal end, where the shaft comprises a lumen for a guidewire and/or a saline flush. The catheter further comprises at least one electrode disposed on the shaft for sensing electrical signals in a bipolar or unipolar fashion and applying pacing to a patient's heart, at least one sensor disposed on the shaft for detecting an event relating to the rapid increase in the rate of pressure increase within the left ventricle of a patient; and communication means configured to transmit data received from the electrode(s) and the sensor(s).

### TECHNICAL FIELD

The present invention is concerned with a catheter, and particularly a catheter that may be utilised in a system and a method for detecting dyssynergy resulting from dyssynchrony, a system and method for determining optimal electrode number and positions for cardiac resynchronisation therapy and/or a method and system for measuring time to fusion as a means of determining degree of parallel activation of the heart. Thus, the invention may be used in relation to patient's suffering dyssynchronous heart failure, and more specifically can apply to the identification of patients who are likely to respond to resynchronization therapy, as well as optionally determining optimal locations for placement of electrodes to stimulate the heart. The invention may also be used for patients who have suffered dyssynchronous heart failure.

### BACKGROUND OF THE INVENTION

Cardiac resynchronization therapy (CRT) is consistently provided according to recognized medical standards and guidelines provided by international medical societies in order to treat patients suffering from various conditions such as a widened QRS complex, (left or right) bundle branch block and heart failure. There are some minor differences between the medical guidelines regarding the specific conditions that should occur before CRT is utilized, such as how wide the QRS complex is, what type of bundle branch block is being suffered and the degree of heart failure.

CRT is associated with a reduction in mortality and morbidity; however, not all patients benefit from such therapy. In fact, some patients may experience deterioration after treatment, some experience devastating complications, and some experience both.

In this regard, it would be beneficial to provide a unifying strategy that reduces the number of non-responders to CRT and optimize the treatment of potential responders, and therefore increases the effectiveness of therapy.

One way to identify such responders may be to detect the onset of synergy following pacing, and determining the resynchronisation potential of a patient. For example, such a method for determining the onset of synergy is defined in GB1906064.9, PCT/EP2020/062149, and the national phases that stem therefrom. A proposed treatment may be suggested by determining potential optimal electrode numbers and positions, for example by taking measurements to determine the degree of parallel activation. For example, such a method for determining the degree of parallel activation is described in GB1906055.7, PCT/EP2020/062146 and the national phases that stem therefrom. For a given treatment, the effect may be validated by calculating time to fusion of wave fronts for electrical wavefronts, thereby giving an indication of the degree of parallel activation of the heart. For example, such a method for determining the time to fusion in a heart is described in GB1906054.0, PCT/EP2020/062152, and the national phases that stem therefrom. A general catheter for performing any such methods, and further discussion of these methods is disclosed in GB2016234.3 and PCT/EP2021/078365.

It would be desirable, however, to provide a catheter that is better suited to performing the above methods. Such a catheter may be provided additionally with a data processing module that can additionally process the data received from the catheter to provide a measure of any of the above values, without need for further post-processing of the data.

### SUMMARY OF THE INVENTION

Viewed from a first aspect, the present invention provides a catheter for assessing cardiac function, the catheter comprising
an elongate shaft extending from a proximal end to a distal end, the shaft having a first curve, a second curve, a third curve and a fourth curve, the shaft comprising:
   a lumen for a guidewire and/or a saline flush;
at least one first electrode disposed on the second curve of the shaft configured to, in use, contact the septum of the left ventricle of the patient, the at least one first electrode for sensing electrical signals and applying pacing to a patient's heart;
at least one second electrode disposed on the third curve of the shaft configured to, in use, contact the free wall of the left ventricle of the patient, the at least one second electrode for sensing electrical signals and applying pacing to a patient's heart, the third curve of the shaft having a larger diameter than the second curve of the shaft;
at least one sensor disposed on the shaft for detecting an event relating to the rapid increase in the rate of pressure increase within the left ventricle of a patient; and
communication means configured to transmit data received from the electrode(s) and the sensor(s).

As discussed below, such a catheter may find particular use when determining function of the heart, and particularly when providing measures indicating whether dyssynergy resulting from dyssynchronous electrical activation is present within a patient. When the catheter is suitably positioned in the left heart chamber with electrodes opposing each other at the septum and contralateral wall and the sensor within the chamber, with each heartbeat a voltage gradient is registered between each electrode and a reference electrode. Such a voltage gradient represents electric activation of the heart at the site of the electrode. The time course of activation of the different electrodes determines the degree of dyssynchrony. Further, and following on from the above, the sensor(s) register events related to the onset of synergy, i.e. events that relate to the rapid increase in rate of pressure rise within the left ventricle, which reflects the point where all segments of the heart begin to actively or passively stiffen. The time to this event is compared with electrical activation and the degree of dyssynchrony, and the presence of dyssynergy resulting from dyssynchrony is registered. Whilst herein the rapid increase in pressure of the left ventricle is referred to, the skilled person would understand that such an event could manifest in a more general pressure wave within the heart of a patient. In this way, the catheter may not necessarily be placed within the left ventricle of the patient.

The heart can then be stimulated from one or more electrode. With each heartbeat a voltage gradient is registered between each electrode and a reference electrode, which as described above can represent the electric activation of the heart. The one or more sensor again registers events related to the onset of synergy. The new set of time events may then be compared to the first set of events and the presence or absence of synergy resulting from resynchronization is registered.

Advantageously, with such a system, it may be possible to quickly and efficiently determine such measures for various positions of electrodes. In this way, not only may it be determined if a patient is indeed a potential responder for cardiac resynchronisation therapy, but also the ideal number and positions of electrodes may be quickly determined.

The at least one sensor comprises a pressure sensor, a piezoelectric sensor, a fiberoptic sensor, and/or an accelerometer. Such sensors can find particular use in detecting events relating to the rapid increase in the rate of pressure increase in the left ventricle, as further discussed below.

The stiffness of the elongate shaft may vary along its length between the proximal end and the distal end. In this way the elongate shaft may have a structure that is ideal for safe, quick and easy positioning within the patient's heart. Optionally, the elongate shaft is provided with a stiff proximal end, a middle part which is of an intermediate stiffness, and a flexible tip at the distal end. Again, such a structure provides for a catheter that may be easily and safely manoeuvred within the heart.

In order to provide such a varying stiffness along its length, the elongate shaft may comprise a multi-lumen extrusion having a first cover around the proximal end, a second cover around the middle part and a third cover around the distal end, the first, second and third covers having differing stiffnesses. In this way, the elongate shaft may comprise a single extrusion of a single material extending along the entire length of the shaft, and the stiffness of the extrusion may be changed along its length according to the stiffness of the cover that is provided around the extrusion at certain points. The multi-lumen extrusion may comprise Pebax 63D, the first cover may comprise Nylon 11 or 12 and optionally a braid wire, the second cover may comprise Pebax 35D, and the third cover may comprise Pebax 75D or Pebax 55D. Such a shaft may be easier to manufacture than a shaft that varies in its composition throughout.

The first curve may be a counter clockwise curve having a first diameter, the second curve may be a clockwise curve having a second diameter, the third curve may be a clockwise curve having a third diameter, and the fourth curve may be a clockwise curve having a fourth diameter. The curves here are described as clockwise, and counter clockwise so as to clearly differentiate between the directions of the curves, it would be appreciated that these can be flipped. For example, if the catheter was flipped, then the curves that were previously clockwise, would now run counter clockwise and vice versa. Therefore, a curve that is defined as “clockwise” need not necessarily run in a clockwise direction, but merely that it is a curve in the opposite direction to a curve defined as “counter clockwise”. In this way, it may more generally be said that a “clockwise” curve may be a curve in a first direction, and that a “counter clockwise” may be a curve in a second direction, opposite to the first direction.

The fourth diameter may be smaller than the first diameter, the first diameter may be smaller than the second diameter, and the second diameter may be smaller than the third diameter.

The first curve may be an arc having an angle of 39°±15%, the second curve may be an arc having an angle of 114.5°±15%, the third curve is an arc having an angle of 76.4°±15%.

The first diameter may be 5 cm±15%, the second diameter may be 6 cm±15%, the third diameter may be 7.5 cm±15% and the fourth diameter may be 1.5 cm±15%.

The catheter may be provided with a first thickness of the catheter towards the proximal end, a second thickness, and a third thickness at the distal end. The first thickness may be greater than the second thickness, the second thickness may be greater than the third thickness. The first thickness may be a diameter of 6 Fr, the second thickness may be a diameter of 5 to 6 Fr, and the third thickness may be a diameter of 3.5 to 5 Fr. As would be appreciated, the diameter describing the “thickness” of the catheter refers to an outer diameter of the cross section of the catheter, as opposed to the diameters that are used to describe the curves of the catheter (that refers to the diameter of the arc). Such a variation in thickness along the catheter may facilitate easier positioning of the electrodes within the heart.

The at least one first electrode may comprise two electrodes disposed on the second curve. The at least one second electrode may comprise at least one electrode on the third curve, and at least one electrode on the fourth curve.

The at least one second electrode may comprise at least two electrodes on the third curve. The least one sensor may be disposed on the second curve.

In this way, the curves of the elongate shaft may vary along its length between the proximal and distal end, a first counter clockwise curve before a larger clockwise curve and a clockwise curved tip with the smallest curve, with the diameters varying for the segmental curves so that the larger diameter curved segments, in combination with the material (i.e. the thickness and/or the stiffness) enables a larger tendency for the catheter to bend, when compared to the smaller diameter curved segments. Also, when a guidewire of a certain stiffness is passed through an inner lumen of the catheter, a resulting stress in the catheter will stretch the arcs of the catheter. This stress provides more stretch in the larger diameter arches, and the total amount of stretch is also determined by the stiffness and thickness of the material of the catheter. A softer and/or thinner portion of the catheter provides less resistance to stretch. In this way, the combination of curve diameter and material/structure of that curve will determine how the guidewire can be used to extend the reach of the catheter, i.e. to extend the distance from the at least one first electrode (the proximal electrode group) to the at least one second electrode (the distal electrode group). This enables the catheter to fit different sizes of failing hearts.

Further, a lumen may be provided such that a guidewire may pass entirely through the catheter. In this way, the guidewire may extend beyond the catheter, such that it may be passed through the mitral valve and into the left atrium, such that tip of the guidewire may be placed in contact with the myocardium of the left atrium, and then utilised to apply an electrical stimulation pulse through the guidewire for artificial stimulation/pacing of the left atrium. Of course, there may be more than one lumen provided within the catheter.

Further, the combination of different directions of the curves enables an effective transfer of torque from the proximal shaft into the distal curve which enables improved, easier and safer positioning of the electrodes within the left heart.

The at least one electrode may comprise a plurality of electrodes disposed along the shaft such that, in use, at least two electrodes may be positioned opposing each other in the heart of the patient. Optionally, the at least one electrode is configured to be placed in contact with the septal wall of the patient (and also to enable recording conduction system biopotentials), and at least one electrode is configured to be placed in contact with the contralateral wall of the patient to enable the measurement of a recorded delay of activation from septum to lateral wall.

In a second aspect, there is provided a system comprising
the catheter as described above;
a signal amplifier;
a stimulator; and
a data processing module;
wherein the catheter is configured to be in signal communication with the stimulator, the amplifier and data processing module such that the electrode(s) and sensor(s) may provide sensed data to the data processing module for further processing, and the electrode(s) may provide pacing to the patient's heart.

Such a system may be utilised to quickly and easily determine how moving the catheter about the heart, and therefore moving the attached electrodes effects the functioning of the heart, and particularly whether pacing makes any marked difference in reducing dyssynchrony and/or dyssynergy.

The data processing module is configured to determine a characteristic response relating to the onset of myocardial synergy from the event relating to the rapid increase in the rate of pressure increase within the left ventricle of a patient.

The sensor(s) may be any kind of appropriate sensor, or a combination of appropriate sensors, such as an acceleration, rotation, vibration and/or a pressure sensor. The sensor(s) may be configured to provide data regarding the pressure within the heart to the data processing module, and wherein the data processing module is configured to filter the pressure data to identify the characteristic response relating to the onset of myocardial synergy. The characteristic response may comprise the beginning of a pressure rise above the pressure floor in a pressure signal filtered above the first harmonic of the pressure signal. The characteristic response may comprise the presence of high frequency components (above 40 Hz) of the pressure signal. The characteristic response may comprises a band-pass filtered pressure trace crossing zero. By filtering the pressure trace it is possible to remove associated noise and more accurately and reliably determine a point that relates to the onset of myocardial synergy. As would be appreciated, a filtered pressure trace or filtered pressure signal as referred to herein can equally refer to a filtered “raw” pressure trace, or any filtered derivative of the raw pressure trace.

Additionally or alternatively, the sensor(s) may be configured to provide acceleration data from within the heart to the data processing module, and the data processing module may be configured to filter the acceleration data to identify a characteristic response relating to the onset of myocardial synergy. For example, the data processing module may be configured to calculate a continuous wavelet transform of the acceleration data to identify a characteristic response relating to the onset of myocardial synergy. The data processing module may be configured to calculate the center frequency of the continuous wavelet transform, wherein the characteristic response is the peak of the center frequency. The data processing module is configured to average the center frequency over a number of heart cycles. By filtering the acceleration trace it is possible to remove associated noise and more accurately and reliably determine a point that relates to the onset of myocardial synergy.

As would be appreciated, in addition or as an alternative to the above, there are provided several further methods herein that enable a characteristic response relating to the onset of myocardial synergy to be determined. The data processing module may be configured to perform one or more of such methods.

For example, the increase of pressure within the heart (for example, pressure within the left ventricle) over time for two different stimuli may be compared. For example, a pressure curve that results from the pacing of the right ventricle and a pressure curve that results from biventricular pacing may be compared. The pressure rises resulting from the two stimuli may be fitted together relative to their stimulation timing, and the pressure level adjusted to fit the diastolic portion of the curves prior to ventricular pacing. The point at which the pressure curve resulting from the stimuli begin to deviate from one another may then be detected, which indicates the time of the onset of synergy of the stimuli that results in the earliest pressure rise.

The portion of the pressure rise curve that follows the time of the onset of synergy on the pressure curve resulting of the stimulus that results in an earlier pressure rise may then be shifted so as to fit on the portion of the pressure rise curve of the stimulus that results in a comparatively delayed pressure rise. The point on the pressure rise curve of the stimulus that results in a comparatively delayed pressure rise at which the curve following the onset of synergy of the stimulus that results in the earlier pressure rise is the point of onset of synergy in the delayed pressure rise curve. The delay may then be calculated between the two determined points of onset of synergy. From such a calculation, a recommendation may be made to which pacing regime should be following in an implanted pacemaker.

The above process may be automated and for the data resulting from any number of pacing regimes/stimuli, whether by a simple matching of the curves (for example, by the fitting of a template to the pressure trajectory with a least squares method) or by a comparison of the mathematical formulae that represent the curves. In this way, an explicit plotting of the pressure curve and a visual matching of the curve may not be necessary, but rather the raw data may be analysed so as to allow for similar conclusions to be reached.

In this way, there can be an automatic detection in the data of the exponential pressure rise, up to the peak dP/dt which results from the onset of synergy. There may be an automatic calculation of the exponential formula that fits the pressure curve, and the time when the exponential formula fits one of a number of curves can be determined.

There may be a template match, and there may be calculated a time offset between the exponential formula and the template matches, or equally a cross-correlation between other measures.

The above method may equally be performed using filtered pressure measurements.

Additionally or alternatively, an advancement of the onset of synergy may be detected by an advancement of the zero-crossing of the band-pass filtered (e.g. 4-40 Hz) pressure curve (Tp) with stimulation from a certain pacing regime compared to another kind of pacing. Such data may be used to indicate the presence of synergy with a certain pacing regime, and therefore that it may be desirable to undergo CRT with that pacing regime.

The method may include calculating a baseline interval (B) by determining a time period between intrinsic atrial activation (Ta) and the associated zero crossing of the resulting pressure curve (Tp). A corresponding time period (Tp1) may be calculated following pacing from a first electrode at a set pacing interval (PI1) after Ta, and the pacing interval reduced until the Ta to Tp interval is less than B. A corresponding time period (Tp2) may be calculated following pacing from a second electrode at a set pacing interval (PI2) after Ta, and the pacing interval reduced until the Ta to Tp interval is less than B. A corresponding time period (Tp3) may be calculated following pacing from the first and second electrodes at a set pacing interval (PI3) after Ta, where PI3 is the same time interval of the lower of PI1 and PI2. By determining which pacing has the shortest corresponding time period Tp, the pacing regime that leads to the highest degree of synergy may be identified.

The data processing module may be configured to identify reversible cardiac dyssynchrony by identifying a shortening of a delay to onset of myocardial synergy as a result of pacing. Specifically, the data processing module may be configured to identify reversible cardiac dyssynchrony of a patient using the at least one sensor to measure the time of the event relating to the rapid increase in the rate of pressure increase within the left ventricle of a patient by identifying the characteristic response in the data received from the one or more sensors, the event relating to the rapid increase in the rate of pressure increase within the left ventricle being identifiable in each contraction of the heart.

The data processing module may be configured to measure the time of the event relating to the rapid increase in the rate of pressure increase within the left ventricle by;
processing signals from the at least one sensor to determine a first time delay between the measured time of the identified characteristic response relating to the rapid increase in the rate of pressure increase within the left ventricle and a first reference time;
   comparing the first time delay between the measured time of the identified characteristic response relating to the rapid increase in the rate of pressure increase within the left ventricle and the first reference time with the duration of electrical activation of the heart;
if the first time delay is longer than a set fraction of electrical activation of the heart, then identifying the presence of cardiac dyssynchrony in the patient;
following the application of pacing by the at least one electrode and/or other electrodes to the heart of the patient;
calculating a second time delay between the identified characteristic response relating to the rapid increase in the rate of pressure increase within the left ventricle following pacing and a second reference time following pacing by:
   using the at least one sensor to measure the timing of the identified characteristic response relating to the rapid increase in the rate of pressure increase within the left ventricle following pacing; and
   processing signals from the at least one sensor to determine the second time delay between the determined time of the identified characteristic response relating to rapid increase in the rate of pressure increase within the left ventricle and the second reference time following pacing;
comparing the first time delay and the second time delay; and
if the second time delay is shorter than the first time delay, identifying a shortening of a delay to onset of myocardial synergy, OoS, indicating that the time period until the point where all segments of the heart begin to actively or passively stiffen has shortened, thereby identifying the presence of reversible cardiac dyssynchrony in the patient.

Further, the data processing module may be configured to determine the degree of parallel activation of a heart undergoing pacing. Specifically the data processing module may be configured to determine the degree of parallel activation of a heart undergoing pacing via a method comprising:
calculating a vectorcadiogram, VCG, or electrocardiogram, ECG, waveforms from right ventricular pacing, RVp, and left ventricular pacing, LVp;
generating a synthetic biventricular pacing, BIVP, waveform pacing by summing the VCG of the RVp and the LVp, or by summing the ECG of the RVp and the LVp;
calculating a corresponding ECG or VCG waveform from real BIVP;
comparing the synthetic BIVP waveform and the real BIVP waveform;
calculating time to fusion by determining the point in time in which the activation from RVp and LVp meets and the synthetic and the real BIVP curves start to deviate;
wherein
a delay in time to fusion indicates that a larger amount of tissue is activated before wave fronts for electrical activation meet, thereby indicating a higher degree of parallel activation.

Further, the data processing module is configured to determine the optimal electrode number and position for cardiac resynchronization therapy on the heart of the patient based on node(s) of a 3D mesh 3D mesh of at least part of the heart with a calculated degree of parallel activation of the myocardium above a predetermined threshold. Specifically, the system may be configured to perform a method determining optimal electrode number and positions for cardiac resynchronization therapy on a heart of a patient, via a method comprising;
generating the 3D mesh of at least part of the heart from a 3D model of at least part of the heart of the patient, or using a generic 3D model of the heart to obtain a 3D mesh of at least a part of the heart, the 3D mesh of at least a part of the heart comprising a plurality of nodes;
aligning the 3D mesh of at least part of a heart to images of the heart of the patient;
placing additional nodes onto the 3d mesh corresponding to a location of at least two electrodes on the patient;
calculating a propagation velocity of the electrical activation between the nodes of the 3D mesh corresponding to the location of the at least two electrodes;
extrapolating the propagation velocity to all of the nodes of the 3D mesh;
calculating the degree of parallel activation of the myocardium for each node of the 3D mesh; and
determining the optimal electrode number and position on the heart of the patient based on the node(s) of the 3D mesh with a calculated degree of parallel activation of the myocardium above a predetermined threshold.

The catheter may be configured to be provided into a patient's heart through arterial access, venous access, subclavian access, radial access and/or femoral access such that the electrode(s) and sensor(s), in use, may be provided within the heart of the patient.

### BRIEF DESCRIPTION OF THE DRAWINGS

Certain preferred embodiments will now be described by way of example only and with reference to the accompanying drawings, in which:

FIG. 1 shows an example catheter

FIG. 2 shows a detailed illustration of an example guidewire for use with the catheter of FIG. 1.

FIG. 3 shows how a guidewire is used to manoeuvre the catheter.

FIG. 4 shows various access routes to bring the catheter into the heart.

FIG. 5 shows a cross section of the catheter.

FIG. 6 shows a more detailed view of the structure of the catheter.

FIG. 7 shows a block diagram of system comprising the catheter.

FIG. 8 shows an example analysis that may be performed to acceleration data, so as to calculate a time to onset of synergy.

FIG. 9 shows a graph of example derivatives of Pₜᵣᵤₑ and P_{reading} to show the sensor calibration effect.

FIG. 10 shows an exemplary catheter, along with some example dimensions over which it may extend.

FIG. 11A shows an optimised catheter before and after shape setting.

FIG. 11B shows in more detail the structure of the catheter.

FIG. 12 shows in more detail the curves of the shape set catheter, and the effect of stress on the shape of the catheter.

FIG. 13 shows in further detail the curves of the shape set catheter.

FIG. 14 shows the introduction of the catheter through the aortic valve, and the coronary ostium.

FIG. 15 shows a distribution of properties throughout the catheter.

FIG. 16 shows the insertion of the catheter within the left ventricle.

FIG. 17 shows how the catheter may be manipulated with a guidewire.

FIG. 18 shows further details of the catheter, such as the cross section.

FIG. 19 shows a system for processing data received by the catheter.

### DETAILED DESCRIPTION

The catheter described herein may be used in methods such as those referred to above that may take place both before and during treatment of patients with presumably dyssynchronous heart failure, with a resynchronization pacemaker (CRT) in order 1) identify the presence of an underlying substrate that identifies patients that are likely to respond positively (manifest resynchronization potential present) to, 2) identify optimal locations for placement of pacing leads/electrodes, and 3) validate placement of optimal electrodes and resynchronisation of the heart. Whilst the skilled person would recognise that the detailed rationale and methodology of these measures, for example as set out in the above mentioned applications, an example method in which the catheter may be used is briefly set out herein.

Whist many of the Figures described herein display many example dimensions and materials, it would be understood that these are example measurements and materials, and any suitable measurements and materials may be selected to provide their function.

Patients are currently referred for implantation of a CRT pacemaker based on international guidelines that describe indication criteria. These criteria are based on inclusion criteria in larger clinical trials and, amongst other things, consists of symptoms of heart failure, reduced ejection fraction (heart function) and a widened QRS complex (preferably left bundle branch block) beyond 120-150 ms. However, currently only 50-70% of patients with one or more indications for treatment with a CRT actually respond to treatment. Reasons for these non-responders are multiple, but lead position, the underlying substrate (dyssynchrony), scar and fibrosis and electrode positions are the most prominent reasons. By improving the detection of the underlying substrate that indicates dyssynchronous heart failure, it is possible improve the selection of responders (in a diagnostic capacity) for optimization of treatment (allowing therapy to be personalized to the patient).

Firstly, it is desirable to detect and define the underlying substrate (resynchronization potential) that defines whether a patient will respond to CRT, and whether the substrate is present or not in patients with standard inclusion criteria. When the substrate is present, one should proceed implantation of a CRT pacemaker, but when the substrate is not present one should follow other guidelines that apply.

When underlying substrate is present, or even if the underlying substrate has not yet been identified, an optimal position for the leads may be found, based on measures of parallelity, which takes scar and fibrosis into account. The measurement of parallelity is performed with guidewires or leads with electrodes inside the heart (for example, in veins or chambers of the heart). Optimal positions are for the placement of the electrodes is then suggested.

When the leads are in optimal position, according to the determined optimal position taking into account the measured parallelity from each node, it is then possible to confirm the response (by either direct or indirect measurements of onset of myocardial synergy), or alternatively reject the position.

If the desired response is confirmed, then a CRT pacemaker should be implanted. If the response is not confirmed, the mapping and measurements of parallelity should be refined before final confirmation. If response is not able to be confirmed, the implantation should be abandoned and known guidelines should be followed for alternative implantations.

It is envisaged that all of the methods referred to above and herein may be used together, or equally may be used separately. In this regard, it is possible to detect the presence dyssynchrony and resynchronization potential, and confirm resynchronization without selecting the optimal lead position, and equally, it is possible to select optimal lead position without confirming underlying substrate and resynchronization.

Therefore, a system may be provided that includes connection to electrodes that allow visualization of signals from the patient and measurements time intervals. Alternatively or additionally, a system may also be provided that includes sensors and electrodes and allows visualization of a heart model and calculations based on the heart model's geometry. Both of the above systems can be combined in the operating room.

An implementation of the above systems and methods will be further described herein by way of an example implementation during surgery.

A patient is firstly taken in to the operating room and sensors and electrodes are fixed on the patient's body surface.

In order to determine the delay to onset of myocardial synergy (OoS), one or more additional sensors may be utilized. For example, one or more of a pressure sensor, piezo-resistive sensor, fibreoptic sensors, an accelerometer, an ultrasound and a microphone may be utilized. Measurements from the additional sensors may be taken in real-time and be processed on location. If the delay to onset of myocardial synergy is short relative to the QRS complex or short in absolute values (for example either shorter than 120 ms or less than 80% of the QRS duration), then the implantation of a CRT device should not occur. When the delay to onset of myocardial synergy is measured to be long compared to the QRS complex or long in absolute values (for example either longer than 120 ms or longer than 80% of the QRS duration), then implantation of the CRT device should occur.

Body surface electrodes are used to determine parallelity (the degree of parallel activation of the myocardium) by collecting surface potentials for an inverse solution ECG activation map of the heart as described above to determine propagation velocity, and thereby the presence of dyssynchrony. Additionally or alternatively, electrodes implanted within the patient's heart may also be used to produce electrical activation maps, and thereby determine the presence of dyssynchrony. If the sensed activation pattern indicates too slow propagation through the tissue, or the inability to provide sufficient parallel activation in the presence of scar tissue, the implantation of a CRT device should not take place.

The patient is then prepared for surgery and sterile draped. Surgery is started as usual and leads are placed in the patient's heart through a skin incision below the left clavicle and puncture of the subclavian vein. The leads are then moved into position in the right atrium and right ventricle.

Dyssynchrony may then be introduced by pacing the right ventricle, and can be confirmed when measuring the delay of myocardial synergy as discussed above. A sensor may be placed in the left heart chamber, or in the right heart chamber, in order to determine the delay of onset of myocardial synergy. In this way, the same calculation may be performed as previously utilized in order to calculate the delay to onset of myocardial synergy.

Once the leads are in position, the coronary sinus is cannulated and an angiography in two planes are performed to visualize the coronary veins.

Once the coronary vein is visualized, cannulation can be performed with either a thin guide wire with an electrode at the tip, or any catheter with one or multiple electrodes for mapping purposes. Measurements of time intervals are then used to characterize one or more of the intrinsic activation, tissue properties and vein properties. The coronary anatomy is then reconstructed in software, and measurements are assigned to positions in the heart model relative to the reconstructed coronary sinus vein.

This data may then be used, in a method performed outside of the body, to calculate parallelity in order to highlight the electrode positions with the highest value of parallelity. Based on these measurements, the surgeon is advised to position the left ventricular (LV) lead with electrodes in a desired position/vein. Similar advice can be given also to reposition the right ventricular (RV) lead. Based on the acquired measurements and the processing thereof, advice can also be provided to include other and/or further electrodes to achieve a higher degree of parallelity. Other electrodes refer to other electrode positions than those available (endocardial, surgical access), and further electrodes refers to the use of multiple electrodes (more than two).

As a result of the above, the coronary vein branches are now seen in two planes and a suitable vein is selected for placement of a left ventricular lead.

When the LV electrodes are in position, the sensors may be used to determine the delay to onset of myocardial synergy, when pacing both the RV and the LV. Different electrodes may be analyzed by repositioning the LV lead at different positions. Measurements of the delay to myocardial synergy may occur using one or more of a pressure sensor, piezo-resistive sensor, fibreoptic sensor, an accelerometer, an ultrasound or by measured bioimpedance (when connected to the RV and LV leads). If the delay to myocardial synergy is not shortened, at least to less than for example 100% of the intrinsic measured value or when the bioimpedance measurements indicate by paradoxical movements that resynchronization is not taking place, the proposed lead positions should be abandoned. The intrinsic value measured from the QRS onset does not include the time from the onset of pacing to ventricular capture, and hence is by definition shorter than that measured from the stimulus. 110% would therefore approximate the time interval measured with intrinsic activation. In this way, the intrinsic delay to onset of synergy measured from the QRS complex can be calibrated by adding, for example, 15 ms to the value reflecting the time from pacing spike onset to electrical tissue capture that occur when artificially pacing.

When pacing the RV, the LV or both, a VCG can be reconstructed and the time to fusion can be calculated. The time to fusion may further be used in order to confirm the already measured parallelity. Surface electrodes can be used for inverse modelling to measure time to fusion. If the measured time to fusion, and the measured parallelity does not concur, the causes of such a discrepancy should be further reviewed.

It is possible that LV leads with multiple electrodes can be used on the discretion of the physician. The use of multiple electrodes can be used in measuring parallelity, and when found to increase parallelity, such an increase in parallelity can be confirmed using time to fusion, and by measuring the delay to onset of myocardial synergy.

Once the lead is in desired position, wherein the delay to onset of myocardial synergy is less than (for example) 110% of initial intrinsic value and less than (for example) 100% of the biventricularly paced QRS complex and, the CRT may be implanted and the device generator connected and implanted in a subcutaneous pocket. If the lead is found not to capture the myocardium or if the location is determined suboptimal based on scientific empiric data or measured intervals (QLV), the lead is repositioned and retested before the device generator is connected. The skin incision is then sutured and closed.

The systems described above may be embodied in an overall system that contains a signal amplifier or analogue digital converter (ECG, electrograms and sensor signals), a digital converter (sensor signals), processor (computer), software, connector to x-ray (either by direct communication with a dicom server or PACS server, or indirect with a framegrabber and an anglesensor). It is possible to use the system with different sensors at user discretion. Further, the system may also be used to solve other problems as well. For example, the systems may be utilized for identification of His region and placement of a pacing lead in the His bundle, with additional measurement of the delay to onset of myocardial synergy.

### Example System

Provided herein is a catheter with a configuration that can be used to detect dyssynergy caused by dyssynchrony, as well as to help select the right patient for therapy. Such a catheter may find particular use in the general method that is outlined above, by which a patient can be identified as a responder for CRT, an ideal pacing regime can be selected, and the response to treatment can be confirmed.

The catheter may comprise a cardiac catheter with a lumen for guidewire and saline flush. The catheter comprises one or more sensors. For example, the catheter may comprise vibrations, pressure, acceleration, and electrodes for sensing electrical local and global cardiac signals. The catheter can be placed in the left or right heart chamber through venous or arterial access, and/or in the coronary vein. Electrodes can be used for sensing electrical signals in a bipolar or unipolar fashion (to a reference electrode on the catheter, or any other electrode connected to the patient body), and the electrodes can be used for pacing the heart at various positions. The catheter connects to a system for processing of the data, either through cables or wirelessly. A guidewire can be passed through the lumen of the catheter to increase the diameter of the distal curve, and a guidewire can be passed through the end of the lumen to get in contact with the cardiac tissue and be used as a sensing and pacing electrode.

When the catheter is passed into the heart chamber, it is possible to use the electrograms provided from the sensors of the catheter to measure the electrical delay from one electrode to the other (or to an electrode that is external to the catheter), and as such determine the electrical activation time. Additionally, using the catheter, it is possible to measure other factors such as vibrations, pressure and acceleration, and then filter the signals to receive measures that can be used to determine the onset of synergy in the heart. Therefore, the catheter can be used to obtain measurements that can be further used to measure the degree of resynchronization and the resynchronization potential. Equally, the catheter maybe provided as part of a system that, for a given set of electrode positions, can measure all data required to calculate the time to onset of synergy. Therefore, system comprising the catheter may be used to quickly and easily determine the resynchronisation potential of a patient.

Such a catheter may provide several uses. As considered above, the catheter may be used to obtain all measurements to be used to detect the onset of synergy following pacing, and determining the resynchronisation potential of a patient. For example, such a method for determining the onset of synergy is defined above, or in GB1906064.9, PCT/EP2020/062149, and the national phases that stem therefrom. The catheter may find use in taking measurements to determine the degree of parallel activation. For example, such a method for determining the degree of parallel activation is described above, or in GB1906055.7, PCT/EP2020/062146 and the national phases that stem therefrom. Equally, the catheter may be utilised to take measurements to determine the time to fusion in a heart. For example, such a method for determining the time to fusion in a heart is described above, or in GB1906054.0, PCT/EP2020/062152, and the national phases that stem therefrom. The catheter may be provided additionally with a data processing module that can additionally process the data received from the catheter to provide a measure of any of the above values, without need for further post-processing of the data.

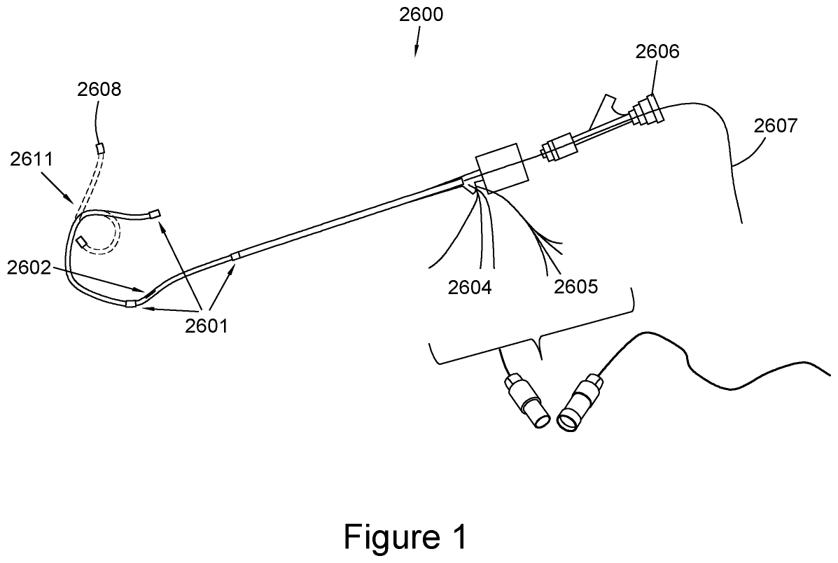

Such a catheter 2600 may be seen in FIG. 1. The catheter comprises one or more electrodes 2601, one or more sensors 2602, a shaft 2603, communication means 2604 and 2605, a hemostatic valve 2606, and a guidewire 2607. The catheter extends to a distal end 2608.

The sensors may be any desired sensor. For example, where the catheter is for use in determining the delay to onset of myocardial synergy, it may be desired that the sensor is a pressure sensor such that it is possible to invasively measure the pressure within the heart, and thereby measure the change of pressure within the left ventricle. Additionally or alternatively, the sensor may comprise a piezoelectric, fiberoptic and/or an, accelerometer sensor. The sensor may detect and transmit events such as cardiac contraction, onset of synergy, valve events, and pressure to a receiver connected to a processor.

The distal end 2608 of the catheter 2600 is a floppy pigtail, such that the electrodes 2601 positioned at the curved distal end may be moved by advancing the relatively stiff guidewire 2607 along the shaft of the catheter. By advancing the guidewire through the catheter 2600, the diameter of the curve provided at the distal end 2608 of the catheter 2600 is increased. This allows for the distal end 2608 of the catheter 2600 to be moved, and thereby allows for movement of the electrodes 2601. Such variable positions are shown in broken lines 2611 in FIG. 1. Additionally, the distal end 2608 of the catheter 2600 may be provided with a soft tip for atraumatic contact with the lateral wall endocardium.

Communication means 2604 may transmit data received from the electrodes 2601, and communication means 2605 may transmit data from the sensor(s) 2602. As shown, these may be provided as physical wires to plug into an external data processing module. Alternatively, they could provide wireless transmission, to transmit the data without a physical connection. The shaft of the catheter 2600 may be of any suitable diameter. For example, the shaft may be a 6 Fr shaft. A saline flush may additionally be provided through hemostatic valve 2606.

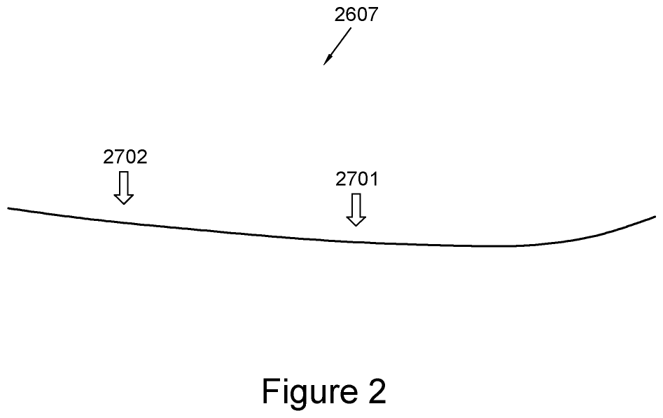

A more detailed view of the guidewire 2607 may be seen in FIG. 2. A stiffer body 2701 is provided at the proximal end of the guidewire 2607, and then a flexible tip 2702 is provided at the distal end. Such an arrangement allows for finer adjustment of the position of the catheter, and the electrodes and sensors that are positioned thereon.

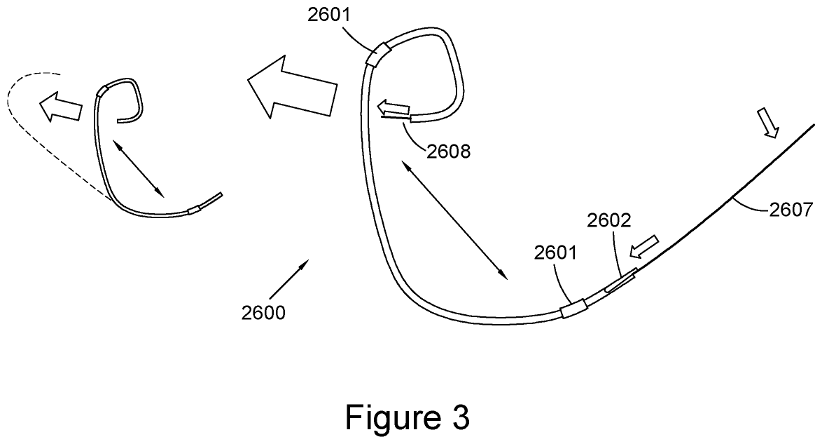

FIG. 3 shows how the guidewire 2607 may be used to manoeuvre the catheter 2600, and more specifically, the electrodes and sensors disposed thereon. As shown, the guidewire 2607 is introduced through the proximal end of catheter 2600. The guidewire extends through the catheter 2600 towards the distal end 2608. As can be seen, the catheter 2600 is a floppy pigtail shape such that when the relatively stiffer guidewire 2607 is advanced through the catheter 2600, the diameter of the curve provided by the catheter 2600 is increased, as seen in FIG. 3. The stiffer body 2701 near the proximal end of the guidewire 2607 provides a more pronounced enlargement of the curve of the catheter than the flexible tip 2702. This provides for more accurate control of the location of the electrodes 2601 (and other sensors 2602) on the catheter 2600.

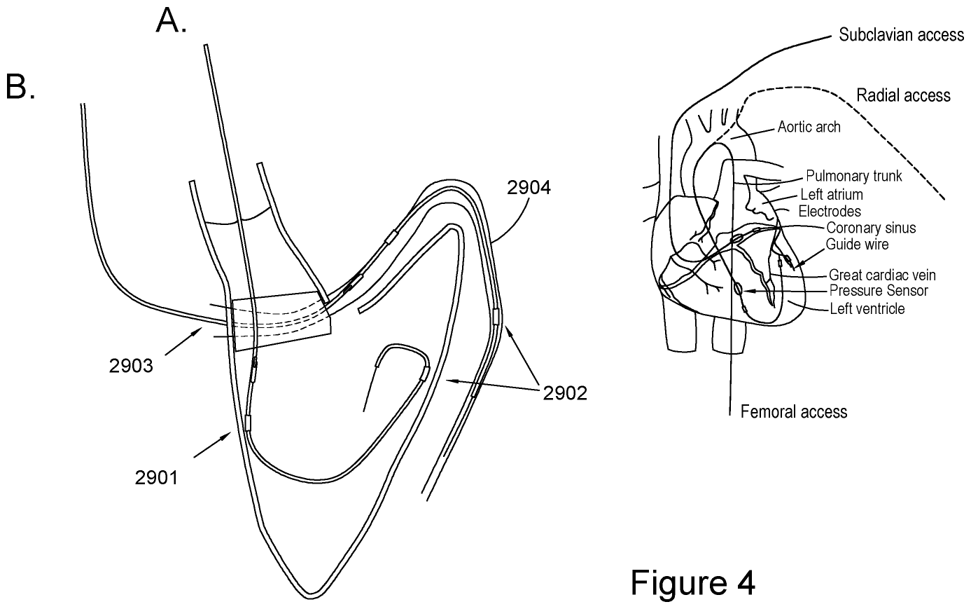

Various different locations within the heart in which the catheter 2600 may be placed are illustrated in FIG. 4. For example, the catheter may be provided through location A, providing arterial access into the heart chamber, or through location B, providing venous access to the heart chamber. Though location A, the catheter (and embedded sensors and electrodes) pass through the septum 2901 toward the contralateral wall 2902, such that electrodes may be placed in the septum and the contralateral wall. Through location B, the catheter may pass through the coronary sinus ostium 2903 and the coronary vein 2904, such that the catheter (and electrode(s)) is passed through the venous system into the coronary vein. Alternatively, the catheter may be provided through subclavian access, radial access or femoral access. The catheter is configured to be positioned in the left heart chamber, with the electrodes opposing each other at the septum and contralateral wall, with and the sensor provided within the chamber. The electrodes are to be provided in contact with the tissue.

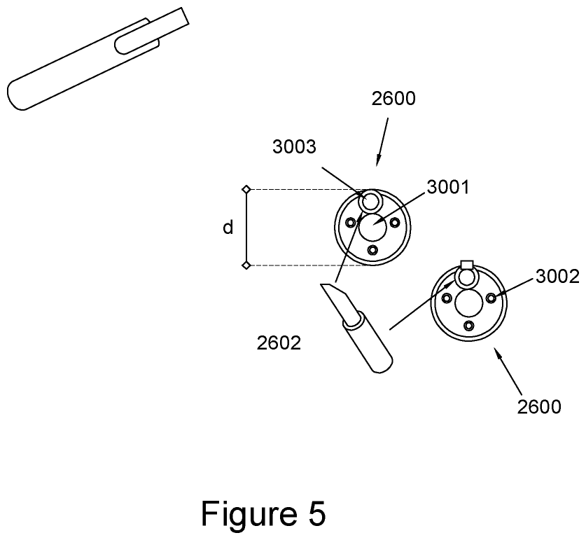

FIG. 5 shows two cross sections of the catheter 2600. As stated above, catheter 2600 may be provided at any suitable diameter d, such as 5 Fr. The catheter 2600 is provided with at least one interior lumen 3001 through which the guidewire may pass. Additionally, saline flush may be provided through the interior lumen 3001. Interior lumen again may be provided with any suitable diameter, such as 0.635 mm (0.025 inches). Catheter 2600 is additionally provided with a number of channels 3002 for electrode leads, and a number of channels 3003 for sensor leads, connected to embedded sensor 2602.

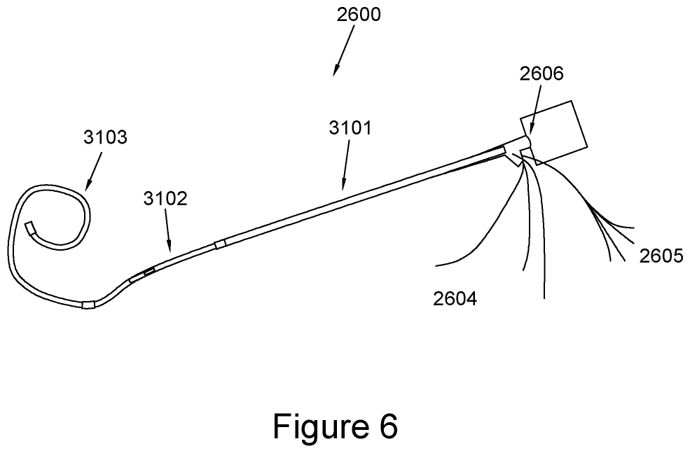

A more detailed view of the structure of the catheter 2600 is seen in FIG. 6. As described above, saline flush may be provided through hemostatic valve 2606. The catheter 2600 is provided with a stiff proximal end 3101, a middle part 3102 which is of an intermediate stiffness, and a flexible tip 3103 at the distal end of the catheter.

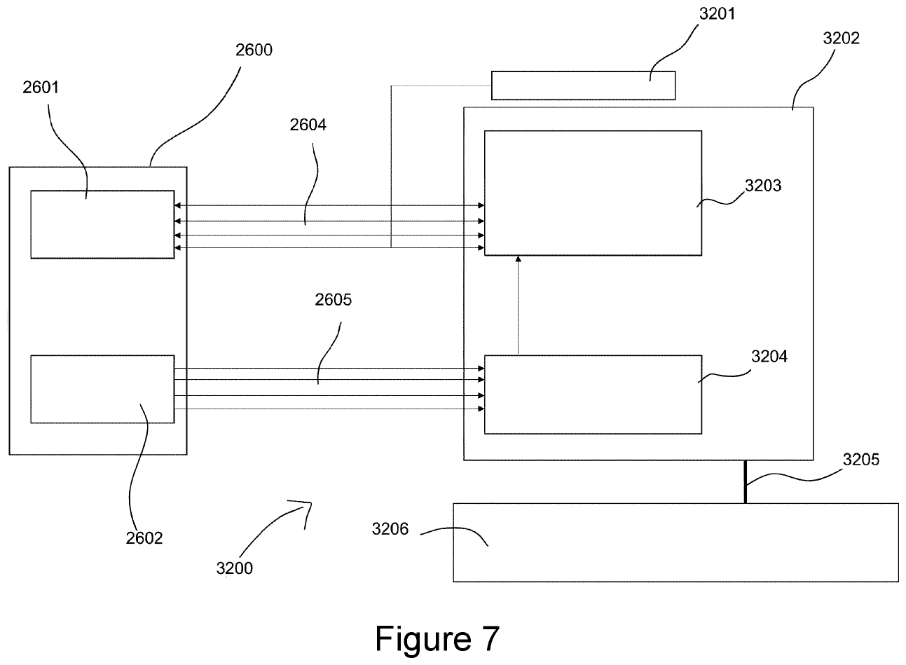

FIG. 7 shows a system 3200 for sensing and processing data comprising a catheter, such as one of the catheters as described herein. The catheter 2600 is in signal communication with stimulator 3201, amplifier 3202 and processor 3206. As described above, catheter 2600 comprises electrodes 2601 and sensor(s) 2602. The electrodes are in signal communication with stimulator 3201 and analog converter 3203 of amplifier 3202 through communication means 2604. The sensor(s) 2602 are in signal communication with receiver and converter 3204, and additionally to analog converter 3203 of amplifier 3202. The amplifier 3202 then provides an output to a processor 3206. For example, the amplifier 3202 may be connected to the processor 3206 by means of a fiber optic cable 3205.

The processing module 3206 may be configured to take the data gathered by the catheter 2600 and further process the data so as to provide meaningful assessments as to the cardiac function of the patient. For example, the data processing module may be configured to calculate the delay to onset of synergy, the time to fusion or a measure of parallelity of the heart of the patient.

For example, the catheter may be provided with at least one piezo-electric sensor 2602 (and/or optical sensor 2602, and/or accelerometer 2602) that is configured to directly measure pressure within the heart. Utilising such information, the catheter 2600 and the processing module 3206 may be configured to automatically and reliably detect a point relating to the onset of synergy, which is distinct from and occurs at some point between the pre-ejection interval (PEI) and electromechanical delay (EMD).

For example, whilst this may be relating to a rapid pressure rise originating from the onset of synergy, the point of the onset of synergy may be better and more reliably represented by filtered pressure traces. As would be appreciated by the skilled person, filtered pressure traces may refer to a filtered raw pressure trace, or equally a filtered derivative pressure trace, to any number of derivatives. Therefore, the system 3200, and more specifically the piezo-electric sensors 2602 of the catheter 2600 and processing module 3206 may be configured to detect the pressure change within the heart, optionally calculate a derivative of the pressure change, and filter the pressure traces so as to give an accurate representation of the onset of synergy. This may be achieved by removing the first harmonics of the pressure wave by band-pass filtering at, for example, 2-40 Hz. This curve, as described above, has a linear upstroke that originates from the onset of synergy and that crosses zero at peak dP/dt. Filtering at, for example, a band-pass 2-40 Hz or 4-40 Hz removes the low, slow frequencies that are associated with dyssynergy and the onset of synergy may be seen as the onset of the pressure increase that leads to, or is directly prior to aortic valve opening or maximum pressure.

This change in rate of pressure increase is because of increasing and exponential cross-bridge formation while passive stretched segments tension increase, either because depolarization or because elasticity model reaches its near maximum. Rapid cross bridge formation with isometric or eccentric contraction leads to high-frequency components in the pressure curve frequency spectrum, which reflects onset of synergy. This phase of the cardiac cycle may be seen when filtering LVP with high pass filter above the 1st or 2nd harmonics. The filtered and characteristic waveform has a near linear increase, from onset of synergy to crossing 0, and continues with a linear increase up to aortic valve opening. The line of linear increase reflects the period with synergy, crossing zero at halfway in the phase, which corresponds to peak dP/dt as described above, and onset of synergy is reflected in where this line starts to rise above the floor of the filtered pressure curve or at its nadir. Additionally, the catheter 2600 and processing module 3206 may be configured to utilise high frequency components (above 40 Hz) of the pressure trace to identify the onset of synergy in the mid range filtered (4-40 Hz) signal as the high frequency components identifies the onset of pressure rise prior to zero-crossing.

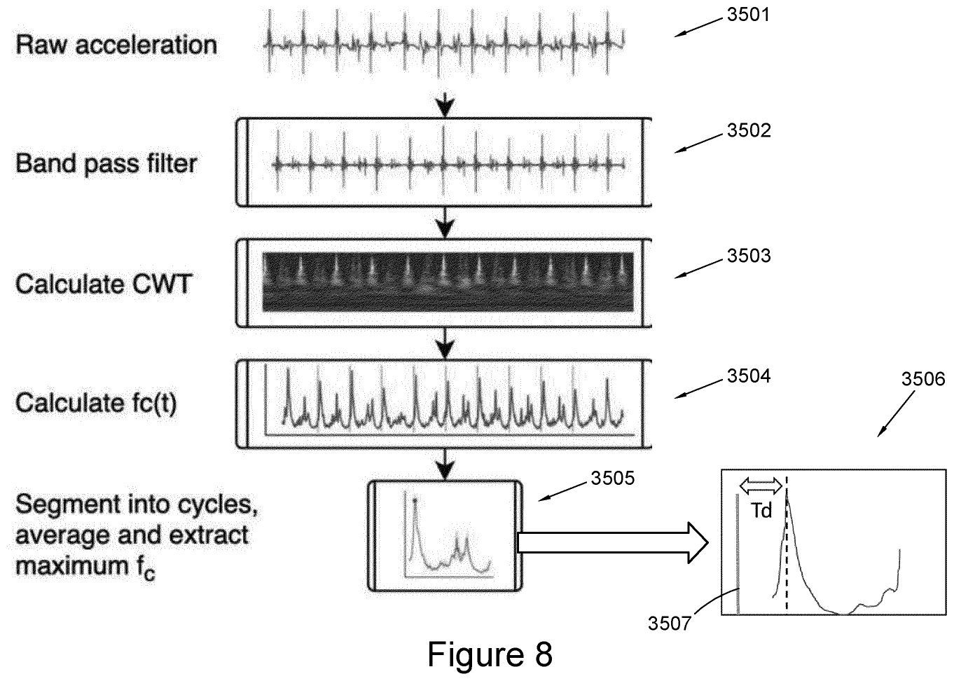

One or more of these points in the pressure trace (the beginning of the linear increase in a band-pass filtered pressure trace, the crossing of zero in a band-pass filtered pressure trace, the onset of high frequency pressure components of the pressure trace), taking data that is filtered from the piezo-electric (or other optical) sensors 2602 of the catheter 2600 may be utilised by the data processing module 3206 to accurately and reliably represent the onset of synergy. Additionally or alternatively, the sensors 2602 may comprise accelerometers that gather accelerometer data within the heart, and from such data determine the onset of synergy, for example as described above and illustrated in FIG. 8. The raw acceleration data 301 may be band pass filtered resulting in data 3502, and from such data, a wavelet scalogram 3503 may be produced, which shows the frequency spectrum over time. The center frequency trace fc(t) 3504 is then calculated from the wavelet scalogram as seen in graph 3504. For each cycle of the heart, averaging each cycle and extracting the time of the peak fc(t), it is possible to determine the time-to-onset of synergy (Td) as seen in graph 3506. The time to onset of synergy may be measured from any suitable reference time, such as the QRS-onset, 3507.

As would be appreciated, any of the measures considered above and herein of detecting onset of synergy (or points relating directly thereto) may be combined to provide a more accurate measurement of the onset of synergy and/or how it varies with treatment. For example, a measure of the time of onset of synergy or a point related thereto before/after treatment calculated by filtering pressure data may be compared and contrasted with the point of onset of synergy calculated using raw acceleration data within the heart before/after treatment. In this way, a reduction in the time to onset of synergy (thereby indicating that reversible cardiac dyssynchrony is present) may be validated using more than one measure.

By utilising any of the above measures, the system may therefore, for each position of the catheter and therefore the electrode(s), automatically determine how time until the onset of synergy varies. In this way, the system can give immediate (or near immediate) feedback on the efficacy of various electrode placements in reversing dyssynchrony and dyssynergy.

In one example, as a representation of the time of onset of synergy, the zero crossing from a filtered signal or a template match from a filtered signal may be detected within a timeframe from a reference time. For example, the zero crossing within a timeframe of ±40 ms of QRSend (so as to ensure that the first zero crossing, being the zero crossing associated with the same heartbeat) is measured. Alternatively, the onset of synergy may be indicated by the timing of the nadir (i.e. the point of pressure increase from the pressure floor) together with high frequency components. As would be appreciated, both of these measures (and others) can represent the onset of synergy, being the point where all segments of the heart begin to actively or passively stiffen. This is practically manifested in the beginning of the rapid pressure rise within the heart.

Whilst the point of onset of synergy is manifested in the increase of pressure within the left ventricle due to the point where all segments of the heart begin to actively or passively stiffen, it will be appreciated by the skilled person that this point can also indirectly be measured in other positions. In this way, and in addition to positioning within the left heart chamber, the catheter may for example be positioned within the coronary veins or in the right heart chamber to provide similar measurements indicative of the onset of synergy, with appropriate filtering of the signal.

In sum, it may be said that the catheter measures pressures and/or vibrations, and can subsequently apply different filters for the assessment of the pressure/vibrations, together with the electrical signals detected by the catheter to determine if dyssynchrony is present or not. Whilst a reduction in the delay to onset of synergy (for example, calculated as described above) indicates that dyssynchrony is present, a prolongation of the interval with stimulation when compared to the baseline (i.e. a case with no stimulation) identifies an iatrogenic potential. Such a situation may be detrimental to the patient's health and should be avoided.

Of course, the methods that may be applied by the catheters described herein are not limited to those discussed herein. The catheters herein may provide wider use, for other methods other than those of detecting the onset of synergy, determining optimal electrode positions and numbers, and measuring time to fusion.

Sensor Calibration Effect on dP/dt:

Advantageously, the sensors of the catheter may not require calibration for time events when using the derivative of pressure that relates to the measurement of onset of synergy.

In theory the offset and gain of the pressure signal should not affect the results of when dP/dt=0 or when dP/dt peaks. The offset will not affect when dP/dt=0 or when dP/dt peaks because the derivative of the offset will go to zero. While the gain will affect the value and slope of the pressure sensor signal, the gain will not affect the time the maximum/minimum of the pressure signal occurs (which is when dP/dt=0) or the time the maximum/minimum slope of the pressure signal occurs (which is when dP/dt peaks).

This effect is illustrated by the below simplified example demonstrating how neither the offset nor gain will affect a cyclical pressure signal.

For example, if the true pressure signal was characterized by the equation:

$${P}_{t r u e} = sin ( 60 t )$$

And the catheter had an offset of 100 mmHg, with a gain of 5 times more than the actual signal. Then the pressure signal reading would be characterized by the equation:

$${P}_{r eading} = 5 sin ( 60 t ) + 100$$

Even given the differences in the true pressure signal and the reading pressure signal, the derivative of both equations with respect to time (t) would be:

$$\frac{d ( {P}_{true} )}{dt} = 60 cos ( 60 t )$$

$$\frac{d ( {P}_{r eading} )}{dt} = 300 cos ( 60 t )$$

Whilst the amplitudes of the two dP/dt equations differ, the time when dP/dt=0 and when dP/dt peaks will be equivalent for both equations

$$t = \frac{( 2 n + 1 ) π}{120}$$

and

$$t = \frac{n π}{60}$$

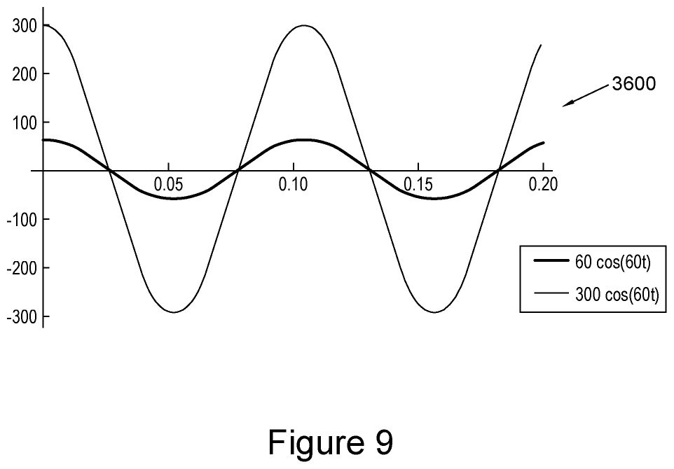

respectively, where n is the value of any integer). This is shown in FIG. 9, which shows a graph of the derivative of Pₜᵣᵤₑ and P_{reading} from the example given above. It can be seen from this example that dP/dt=0 and dP/dt peaks at the same times for both of Pₜᵣᵤₑ and P_{reading}.

It should be noted that signal changes due to temperature, drift, and atmospheric pressure all have a time dependency, which means, in theory these changes may have some effect on when dP/dt=0 or when dP/dt peaks. However, the largest discrepancies caused by temperature and drift will occur when the catheter is first being introduced in the body, as this is when the sensor is transitioning from a dry state at room temperature to a “wet” state at body temperature. By the time the catheter is deployed/positioned and data starts to be analyzed, the amplitudes and frequencies of the changes due to temperature, drift, and atmospheric pressure are all be minimal compared to the amplitude and frequencies of the pressures in the heart. Therefore, even without correcting for changes due to temperature, drift, and atmospheric pressure, the effects to dP/dt=0 or when dP/dt peaks should be negligible.

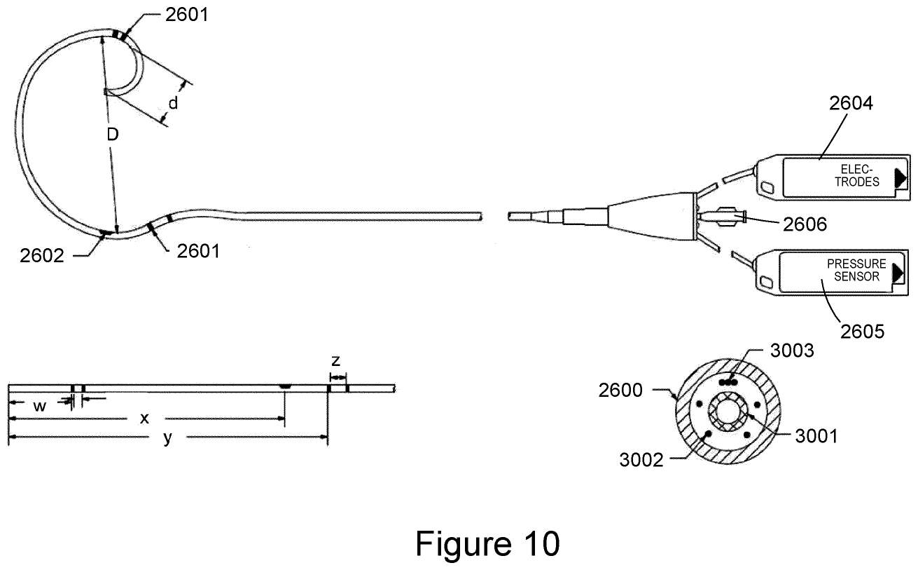

An exemplary catheter is shown in FIG. 10, along with some example dimensions over which it may extend. In order to provide electrodes 2601 and sensors 2602 at desired positions within the heart, the flexible tip may be provided at a small diameter, d. The middle part of the catheter may be provided at a larger diameter, D. As an example, diameter d may be in the order of 1.5 cm, and diameter D may be in the order of 6 cm. The total length of the catheter may be in the order of 130 cm. Electrodes 2601 closest to the tip of the catheter may be 1 mm wide, and may be positioned at a distance w from the tip, for example 3 cm. The two electrodes disposed closest to the tip may be disposed 8 mm apart. Sensor 2602 may be provided at a distance x from the tip of the catheter, for example 11 cm. Further electrodes 2601 may provided at a distance y from the tip of the catheter, for example 13 cm. Said electrodes may be provided at a distance z apart, again this may be for example 8 mm. Of course, said dimensions are exemplary, and other dimensions are envisioned.

In sum, in the above system, the distal segment of the catheter is adapted to be positioned with electrodes opposing each other in the heart. The distal segment has an area intended to contact the heart tissue. The distal segment carries one or more electrodes and one or more sensors (for example a pressure sensor, piezoelectric sensor, fiberoptic sensor, accelerometer) located proximal on the distal end of the catheter. The sensor(s) provide data on cardiac contraction, onset of synergy, valve events, pressure to a receiver connected to the processor. The electrodes connect to an amplifier that connect to a processor. The electrodes connect to a stimulator. The processor may analyse the data received to determine a point relating to the onset of synergy, and utilise this to determine if dyssynchrony and dyssynergy is present, and then further if stimulating the electrodes results in reversal of dyssynchrony and dyssynergy.

When the catheter is suitably positioned in the left heart chamber with electrodes opposing each other at the septum and contralateral wall and the sensor within the chamber, with each heartbeat a voltage gradient is registered between each electrode and a reference electrode. Such a voltage gradient represents electric activation of the heart. Further, and following on from the above, the sensor(s) register events related to the onset of synergy, i.e. events that relate to the rapid increase in rate of pressure rise within the left ventricle, which reflects the point where all segments of the heart begin to actively or passively stiffen to a maximal extent. The time to this event is compared with electrical activation, and the presence or absence of dyssynchrony and dyssynergy is registered.

The heart can then be stimulated from one or more electrode. With each heartbeat a voltage gradient is registered between each electrode and a reference electrode, which as described above can represent the electric activation of the heart. The one or more sensor again registers events related to the onset of synergy. The new set of time events may then be compared to the first set of events and the presence or absence of resynchronization is registered.

Advantageously, with such a system, it may be possible to quickly and efficiently determine such measures for various positions of electrodes. In this way, not only may it be determined if a patient is indeed a potential responder for cardiac resynchronisation therapy, but also the ideal number and positions of electrodes may be quickly determined.

### Optimised System

In view of the above, an optimised catheter and system has been developed in order to more conveniently, and more accurately place electrodes and/or various sensors within the heart, enabling for more accurate determination various measurements, such as the measurement of the Onset of Synergy.

A curve may be discussed herein as having a radius or a diameter. Such a curve may be defined by an arc of a circle having a given radius or diameter. Therefore, as would be appreciated, when a curve is discussed as having a radius or diameter, it would be understood that such a radius/diameter may also be thought as referring to the corresponding radius/diameter of the circle upon which the curve can be defined. As would also be appreciated, any angles and radius/diameter referred to may refer to the angles/diameters of the curves of a “set” catheter, and may vary in use under external stresses. In addition, whilst the curves here are described as clockwise, and counter clockwise so as to clearly differentiate between the directions of the curves, it would be appreciated that these can be flipped. For example, if the catheter was flipped, then the curves that were previously clockwise, would now run counter clockwise and vice versa. Therefore, a curve that is defined as “clockwise” need not necessarily run in a clockwise direction, but merely that it is a curve in the opposite direction to a curve defined as “counter clockwise”. In addition, whilst various dimensions are included in the Figures, it would be appreciated that these are not limiting, and may vary.

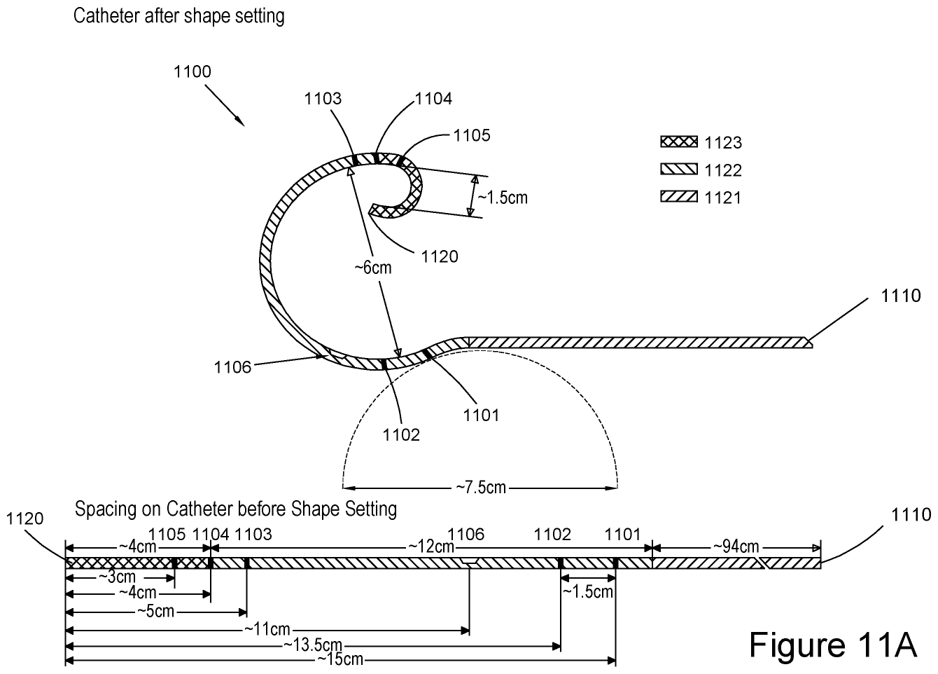

Such a catheter 1100 may have a configuration as shown in FIG. 11A, which shows the shape of the catheter 1100 before and after shape setting (i.e. at its fully straight, maximum length, and the shape in its final use). Some example dimensions of catheter 1100 may additionally be seen in FIG. 11A. As can be seen, the catheter may have a total length of around 110 cm when fully straight, with the dimensions and spacing between features as set out in FIG. 11A. The catheter 1100 extends from proximal end 1110 to distal end 1120.

The catheter has three sections 1121, 1122, 1123 each having different stiffnesses. The proximal section 1121 closest to the proximal end 1110 of the catheter may be the stiffest (for example, comprising Nylon 11 or 12 or materials typically used for intracardiac angiopgraphic catheters like polyamide, polyurethane, Teflon and combinations thereof), and the distal section 1123 at the distal end 1120 may be the most flexible (for example, comprising Pebax 35D). The intermediate section 1122 has a stiffness between the two other sections (for example, comprising Pebax 75D or 55D). The distal section 1123 may extend around 4 cm from the distal end 1120 of the catheter 1100. The intermediate section 1122 may extend around 12 cm from the distal section 1123. The proximal section 1121 may extend the remainder of the length of the catheter, for example, around 94 cm.

As may be seen in FIG. 11B, in order to provide such a variable stiffness, the catheter may comprise a central extrusion 1131 extending throughout all three sections 1121, 1122, 1123. The central extrusion 1131 may comprise one or more lumens extending therethrough. In the example of FIG. 11B, the central extrusion 1131 is a multi-lumen extrusion. Then, in order to provide the different relative stiffnesses between each section 1121, 1122, 1123, the central extrusion may be covered in one or more covers or jackets 1132 of differing material at each section. For example, the central extrusion 1131 may comprise of Pebax 63D, the relatively stiffer Pebax 75D or the relatively softer Pebax 55D, and be the same material throughout. To provide the stiff proximal end 1121, a jacket of a relatively stiff material such as Nylon 11 or 12, with or without braid wire, may be provided around the proximal end of the catheter, thereby stiffening the central extrusion at the proximal end. Then the intermediate section 1122 may be covered by a jacket of a material relatively less stiff than that of the proximal end, such as Pebax 75D or 55D. Finally, the flexible tip 1123 may be covered in an even less stiff material, such as Pebax 35D. By providing such a common central extrusion throughout the catheter, and varying the stiffness by way of outer jackets with varying stiffness, the catheter may be easier to manufacture. As can be seen in FIG. 11B, one or more of the materials of the catheter may be provided with a portion of Barium Sulphate (BaSO₄). For example, the material of one or more of the jackets surrounding the proximal end 1121, the intermediate section 1122, or the distal end 1123 may comprise 20% BaSO₄.

As can be seen in FIG. 11A, catheter 1100 may comprise five electrodes 1101, 1102, 1103, 1104, and 1105. These five electrodes may be arranged in two groups, with three electrodes 1105, 1104, and 1103 arranged in relative proximity close to the distal end 1120 of the catheter, and the remaining two electrodes 1102, 1101 arranged further from the distal end 1120 of the catheter. The distance between electrodes between the two groups of electrodes may be chosen such that at least one electrode of the group of electrodes positioned further from the distal end may contact the septum, whilst at least one electrode of the group of electrodes positioned closer to the distal end may contact the free wall. Additionally, the distal electrodes may have a spacing that is narrower than what is shown. By providing a narrow spacing between at least a pair of the distal electrodes, bipolar sensing may be achieved, thereby improving spatial resolution removing far-field sensing. In this way, the signal received by the electrodes can be used to detect local electrical activation, and thereby secure accurate assessment of electrical delay from a reference (onset of QRS) to that region.

For example, the Euclidean distance between the two sets of electrodes may be in the region of 6.5 cm, that may increase up to 9 cm with a stiff guidewire inserted in a lumen within the catheter.

When a guidewire is inserted in an inner lumen of the catheter, and the catheter is positioned correctly in the heart as seen throughout the figures, then the guidewire may be advanced through the catheter, as the tip of the catheter points in the direction of the left atrium. In this way, the guidewire may pass through the catheter, and then passed through the mitral valve and into the left atrium. When the tip of the guidewire is placed in contact with the myocardium of the left atrium, it is then possible to connect a stimulator to the proximal end of the guidewire, and apply an electrical stimulation pulse through the guidewire for artificial stimulation/pacing of the left atrium.

By way of example, when measured on the catheter before shape setting, electrode 1105 may be disposed around 3 cm from the distal end 1120 of catheter 1100, and therefore in the distal section 1123. Electrode 1104 may be disposed around 4 cm from distal end 1120. Electrode 1104 can therefore either be located on the distal section 1123, the intermediate section 1122, or between the distal section 1123 and the intermediate section 1122. Electrode 1103 may be disposed around 5 cm from distal end 1120, and therefore be disposed in the intermediate section 1122. These electrodes 1105, 1104 and 1103 may form a first group of electrodes, which are disposed closer to the distal end of the catheter 1100 than a second group of electrodes 1102, 1101. Of course, the first and second groups of electrodes may comprise more, or fewer electrodes.

Disposed between the first and second sets of electrodes may be the pressure sensor 1106, which may be disposed around 11 cm from the distal end 1120 of the catheter 1100. Alternatively, the pressure sensor 1106 may be disposed closer to the distal end of the catheter, or at the distal end of the catheter, so as to ease manufacturing.

Electrode 1102 may be disposed around 13.5 cm from the distal end 1120 of the catheter 1100, and electrode 1101 may be disposed around 15 cm from the distal end of the catheter 1100, and therefore both these electrodes may lie in the intermediate portion 1122.

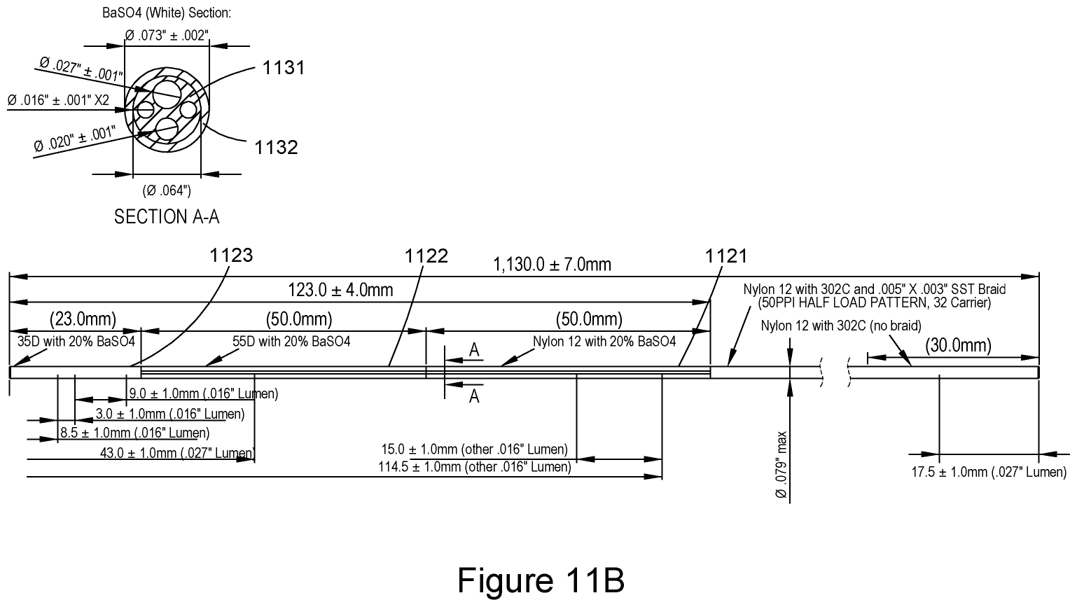

The catheter may then be set in a desired shape, as is best shown in FIG. 12. The catheter 1100 may be described with reference to four segmental curves, curve 1, curve 2, curve 3 and curve 4. Described starting from the proximal end 1110, once set, the catheter may curve counter clockwise to form curve 1, with a relatively small diameter (typically in the range of 4 cm to 6 cm). Then, catheter 1100 may curve clockwise with a larger diameter (typically in the range of 5 cm to 7 cm) to form curve 2, and then begin to curve further clockwise, but with a larger diameter to form curve 3 (typically in the range of 7 cm to 9 cm). Then, the catheter 1100 may curve with a final, tight clockwise curve forming curve 4 having a small diameter (typically in the range of 1-3 cm).

As can be seen in FIG. 12, when the catheter is being introduced to a patient through, for example, arterial access, venous access, subclavian access, radial access and/or femoral access, it may experience a force on the shaft of the catheter 1100. For example, when being introduced through parallel walls, the catheter may contact the walls on curve 2 and curve 3, although the catheter may also contact the walls on curve 4. The resultant force would force the large diameter segment (i.e. curve 3) inwards.

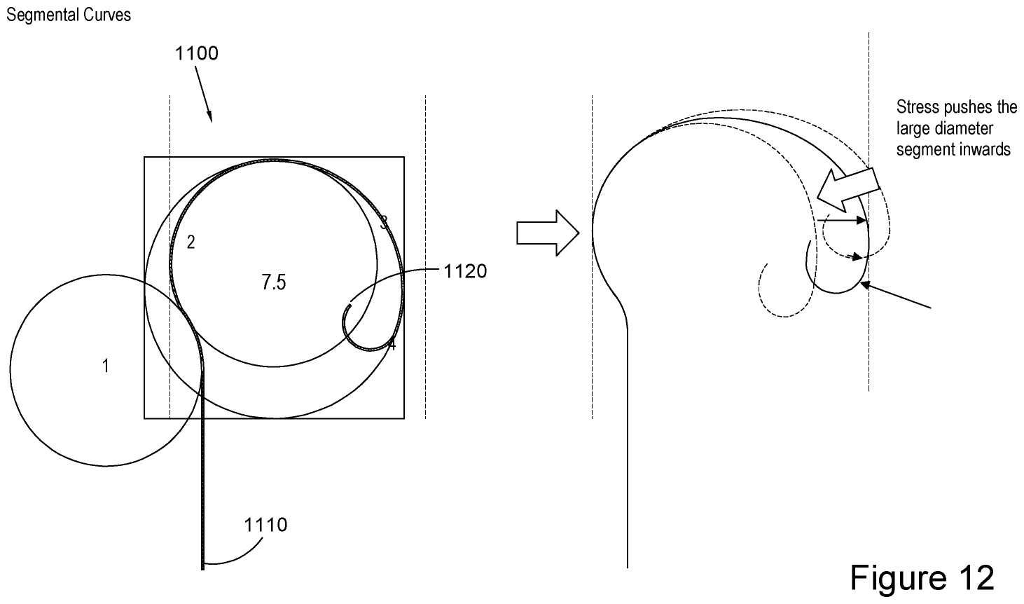

FIG. 13 shows the curves of the catheter 1100 in more detail, and more clearly shows how each curve relates to an arc of a circle having a given radius/diameter. As described briefly above, the first curve (curve 1) closest to the proximal end 1110 bends counter clockwise so as to form a roughly 39°±15% arc of a circle having a radius of around 2.5 cm±15%. Then, the catheter bends clockwise (curve 2), forming a roughly 114.6°±15% arc of a circle having a radius of around 3 cm±15%. The curve then follows another curve (curve 3), also clockwise but with a larger radius of around 3.75 cm±15%, forming a roughly 76.4°±15% arc. Finally, the catheter 1100 ends with a final clockwise curve (curve 4) which has a radius of around 0.75 cm±15%.

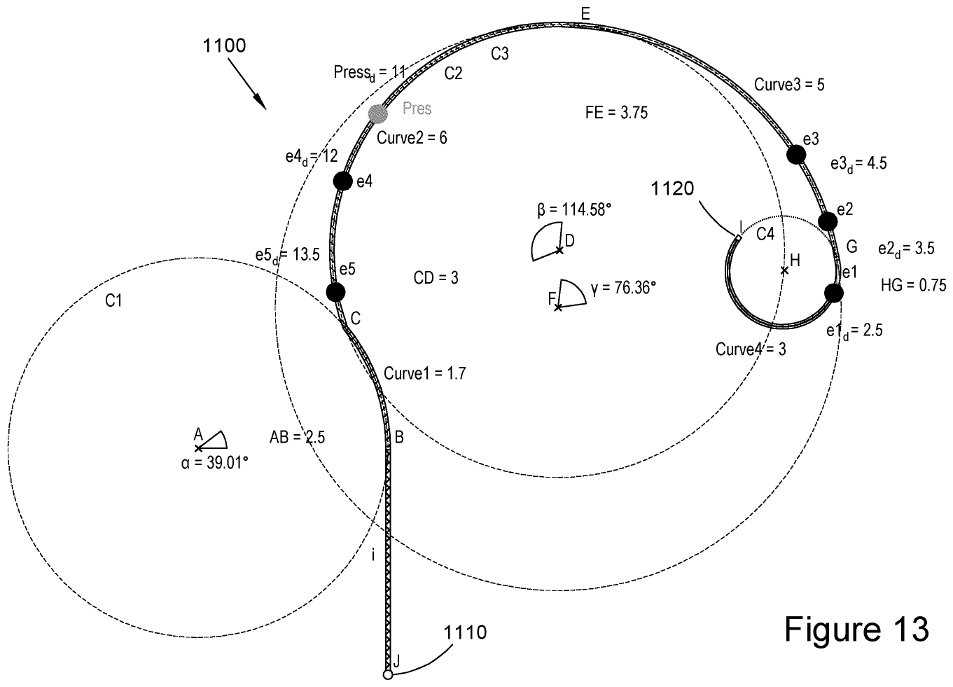

As can be seen in FIG. 14, such a curve setting allows the catheter 1100 to easily be passed through, for example, the aortic valve. This may be achieved either with the distal end curved, and without engaging in the coronary ostia, or alternatively, the large bend (i.e. curve 3 described above), when opposed by a stress (as illustrated and described above) and following along the walls of the ascending aorta, may be rotated by the operator to pass the valves between the cusps, and pushed into the left heart cavity when the valve opens. This is particularly beneficial when providing the catheter within the heart to perform the methods described above, and particularly when determining the onset of synergy.

Specifically, the combination of the different stiffnesses along the catheter, and the presence of one or more lumen(s) through which a guidewire may be passed to provide stiffness to the catheter enable the safe and effective placement of the catheter within the left heart.

Firstly, the catheter may be placed over a wire that has already passed the aortic valve. Secondly, the curve of the catheter is similar to the curve of the aortic arch. In this way, when the curved tip of the catheter is pushed forward against the blood stream (retrogradely) in the aorta it will engage with the aortic valve without entering the coronaries, and it is then possible to pass the valve (with rotation) to align with the opening of the tricuspid (or bicuspid) aortic valve into the left heart. Thirdly, the catheter tip may engage with the aortic valve in such a way that it is pushed upwards antegradely into the ascending aorta in a way that allows the larger curve of the catheter to engage with the valve and to be rotated so that it may pass the valve by alignment with the opening of the valve, as can be seen in FIG. 14.

Once in the left ventricle, the stiffness of the catheter, and the various diameter of the curves may be manipulated by inserting a stiff guidewire, and the catheter can be rotated so as to position the electrodes in diametral positions within the left heart. Such positioning allows for stimulation at different and widely separated sites of the left heart by the electrodes.

In addition, the shape of the catheter enables insertion into the coronary vein and its branches. The shape of the catheter fits the curvature of the coronary vein and, for example, a 0.014″ (0.36 mm) coronary guidewire may be passed through the catheter when the catheter is in the coronary vein. The guidewire may then be used to cannulate a branch of the coronary vein, subsequently thereby enabling the catheter to be guided over the wire into the cannulated branch. The distal electrode(s) may then be used to stimulate the epicardial region of the left heart that is covered by the branch that is cannulated, while the proximal electrode(s) may be used to stimulate the atrium. The pressure sensor may then capture audible signals from heart valve vibrations that can be used to detect the Onset of Synergy, and pacing may be guided to resynchronize the left heart. For example, pacing may be guided by the atrioventricular delay, such that the fusion optimized interval technique may be used, as would be appreciated by the skilled person.

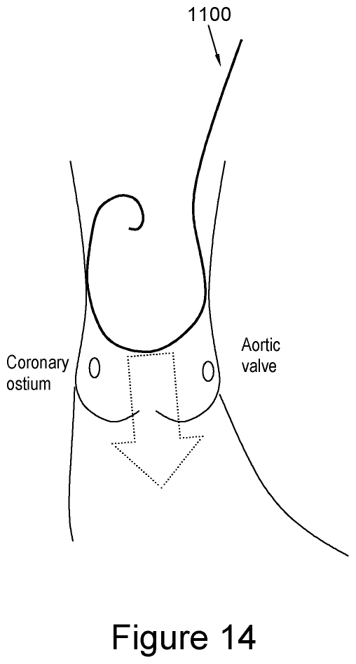

FIG. 15 shows the distribution of various properties across an optimised catheter from proximal end 1110 to distal end 1120. Each property is shown as it is distributed along the length of the catheter. The grid upon which FIG. 15 is set represents a scale, whereby the large squares each indicate a 1 cm increment.

Taking each property in turn, in general, the catheter may be provided with two colours, a first colour extending from the proximal end 1110, and a second colour on the distal tip.

Similarly, the catheter may be provided such that the proximal end 1110 is non-radiopaque, and only the distal end is radiopaque.

The catheter may be provided with a first dimension, such as 6 Fr at the proximal end 1110. Then, the catheter may then get smaller as the catheter extends towards the distal end 1120, to a second dimension such as 5 Fr. Then, at the distal end of the catheter, the catheter may be provided in a third dimension such as 3.5 Fr. As an example, and as shown in FIG. 15, the third dimension may extend for around 2 cm from the distal end 1120, and the second dimension may extend for around 8 cm from the end of the third dimension. The first dimension then extends from the second dimension to the proximal end 1110 of the catheter. Such a variation in diameter (thickness) of the catheter may aid in allowing for the electrodes of the catheter to be placed within the heart in a safe, quick and easy fashion.

Then in the example of FIG. 15, regarding the stiffness, at the proximal end 1110 the catheter may be provided with a braid, which in turn provides a first stiffness. Then, a second stiffness is provided, extending around 3 cm. Extending from the second stiffness is a third stiffness, that extends around 11 cm. Finally, from the end of the third stiffness, a fourth stiffness is provided extending around 2 cm to the distal end 1120 of the catheter. Typically, the fourth stiffness will be less stiff than the third stiffness, which in turn, is less stiff than the second stiffness.

Finally, the curvature of the catheter is illustrated in FIG. 15 is shown. The shaft has at the proximate end 1110 has no curvature. Then, curve 1 extends for a length (i.e. a circumference of an arc) of around 1.7 cm. From curve 1, curve 2 extends for around 6 cm in an opposite direction. Then, curve 3 extends for around 5 cm, ending up in curve 4 which extends for around 3 cm to the distal tip of the catheter. Also indicated here is the relative locations of each electrode, as well as a pressure sensor.

Whilst the above sets out extensively the properties of an example catheter, the skilled person would recognise that the teachings presented herein are not restricted to exactly such dimensions, and equally, each of the above distributions of properties may be found in isolation in an example catheter, i.e. these properties are not inextricably linked to each other.

Rather, the combination of stiffer and/or thicker materials in the sections with the initial curves (i.e. curves 1 and 2 identified above) having lower radiuses, and softer materials with the larger curves (i.e. curves 2 to 3 identified above) enables the improved performance of the catheter, which specifically allows for electrodes to be placed within the heart in a safe, quick and easy fashion, for example, as described above with reference to FIG. 14. Then, by providing a relatively soft, and narrow distal end, it allows for the distal softer tip to be easily extended when a relatively stiffer guidewire is introduced via a lumen to the distal end. This lumen (and associated guidewire) allows for changing of the diameter of the distal curve with greater separation of the electrode groups, and also an extended reach of the distal electrodes when a guidewire is passed through the lumen. The distal softer tip fully extends when guidewire is inserted to enable advancement of the catheter along the guidewire inside the artery/blood vessels. Further, as described above, the guidewire may extend beyond the catheter, such that it may be passed through the mitral valve and into the left atrium, such that tip of the guidewire may be placed in contact with the myocardium of the left atrium, and then utilised to apply an electrical stimulation pulse through the guidewire for artificial stimulation/pacing of the left atrium.

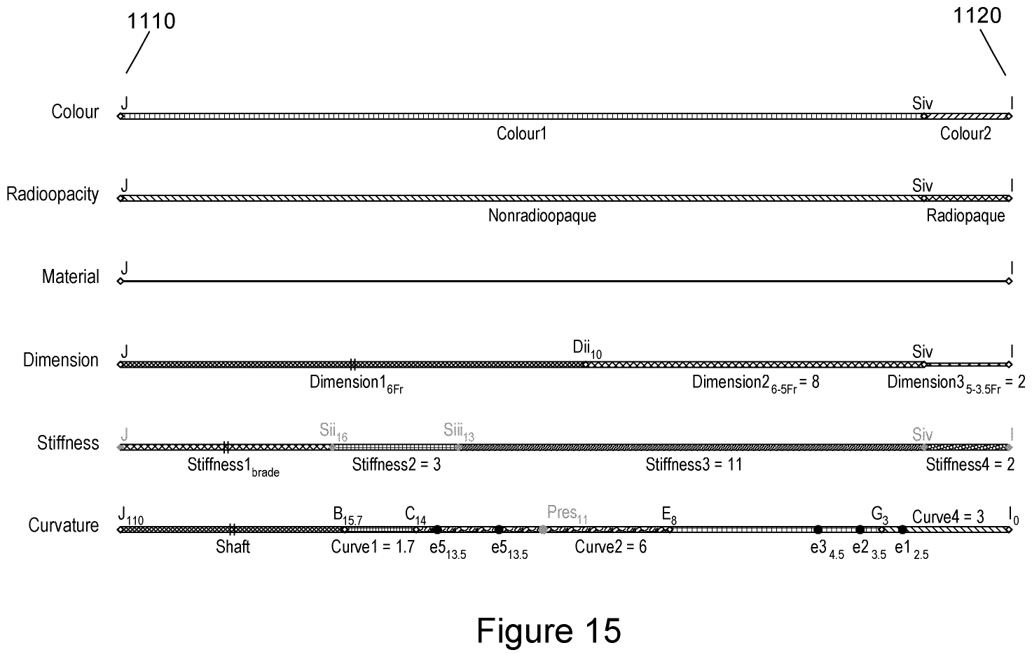

FIG. 16 shows the insertion of the catheter 1100 into a left ventricle having a septum 1610 and a free wall 1620. As can be seen, the shape of the catheter outline above ensures that at least one of electrodes 1101, 1102 contacts the septum, and at least one of the electrodes 1103, 1104 and 1105 contacts the free wall 1620, regardless of whether the ventricle is small, i.e. has a free wall 1621, normal sized, i.e. has a free wall 1622, or if the left ventricle is large, i.e. has a free wall 1623. Additionally, the shape of the catheter allows for the pressure sensor 1106 to be placed in the left ventricle, so as to take the relevant measurements for the methods referred to above.

As discussed above, the catheter 1100 may also be provided with at least one lumen inside the catheter for a guidewire and/or for saline flush. The guidewire can be used to affect the radiuses of the catheter 1100, such that the electrodes 1101, 1102, 1103, 1104, 1105 may be suitably placed within the left ventricle.

When passing through a stiff guidewire with a soft tip (for example, a stiff supportive guidewire with a diameter of 0.014 inches (0.036 cm)), the distal curve with the larger diameter (curve 3) will extend to a larger degree than all curves with a smaller diameter, due to lower susceptibility of a larger diameter curve to remain in its curvature when opposed by a stress, which in this case, is stress applied by the stiffer guidewire. In addition, the insertion of the stiff guidewire through the lumen results in a stress being applied in the catheter, which in turn stretches the arcs of the catheter. This stress provides more stretch in the larger diameter arches, and the total amount of stretch is also determined by the stiffness and thickness of the material of the catheter. A softer and/or thinner portion of the catheter provides less resistance to stretch. In this way, the combination of curve diameter and material/structure of that curve will determine how the guidewire can be used to extend the reach of the catheter, i.e. to extend the distance from the at least one first electrode (the proximal electrode group) to the at least one second electrode (the distal electrode group). This enables the catheter to fit different sizes of failing hearts.

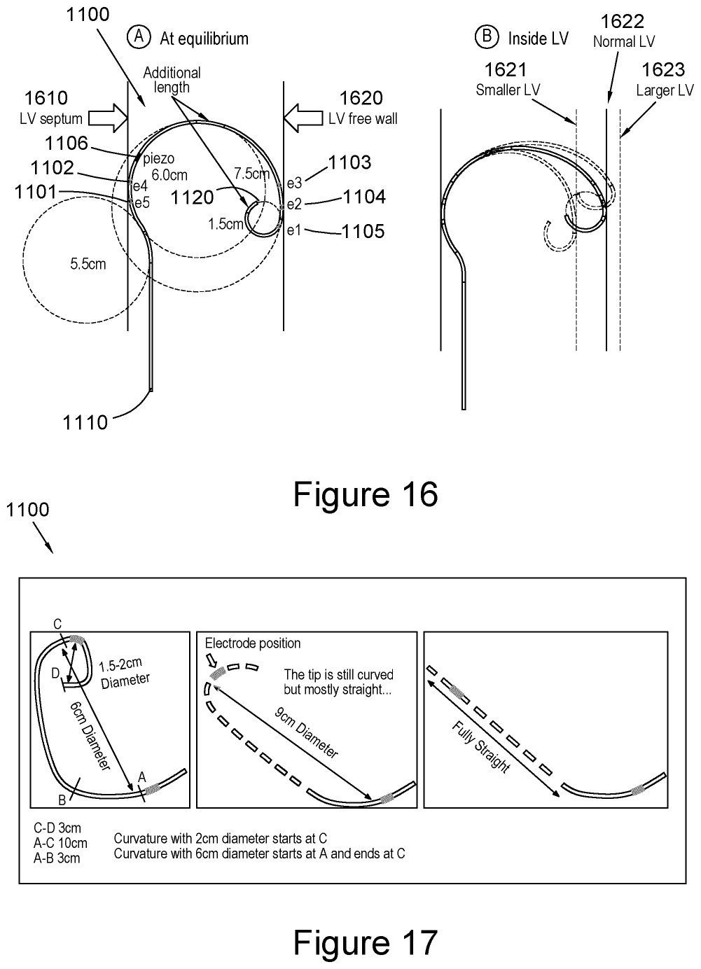

Therefore, the guidewire may increase the diameter of the distal curve (curve 3) and also increase the distance between the proximal and distal electrodes, such that the catheter fits a larger heart diameter, and enables electrode contact for artificial stimulation at two sites for resynchronization, such as the stimulation that may be required to perform one or more of the methods that are referred to above. This effect may be seen in FIG. 17, showing how the shape of the catheter varies as the stiffer guidewire is passed through the lumen.

As illustrated, when inserting a stiff guidewire, the curves will extend (i.e. increase in diameter), and the softer distal tip will straighten, thereby allowing the guidewire to be advanced past the tip of the catheter, and along the vasculature. Then, once the catheter is in the aortic arch, the retraction of the guidewire allows the distal tip to revert back to its shape (i.e. that of curve 4), which prohibits the distal tip from enter into smaller arterial branches of the aortic arch and the ascending aorta, while the general curvature of rest of the catheter 1100 allows the catheter, when pushed, to find its way past the aortic arch and into the descending aorta. Then, when engaging with the aortic valve, the curved catheter tip will either pass through the valve, or move upwards along the aortic wall to position the larger curve towards the valve. At this point, the catheter can be rotated to fit the opening of the aortic valve to be pushed into the left heart.

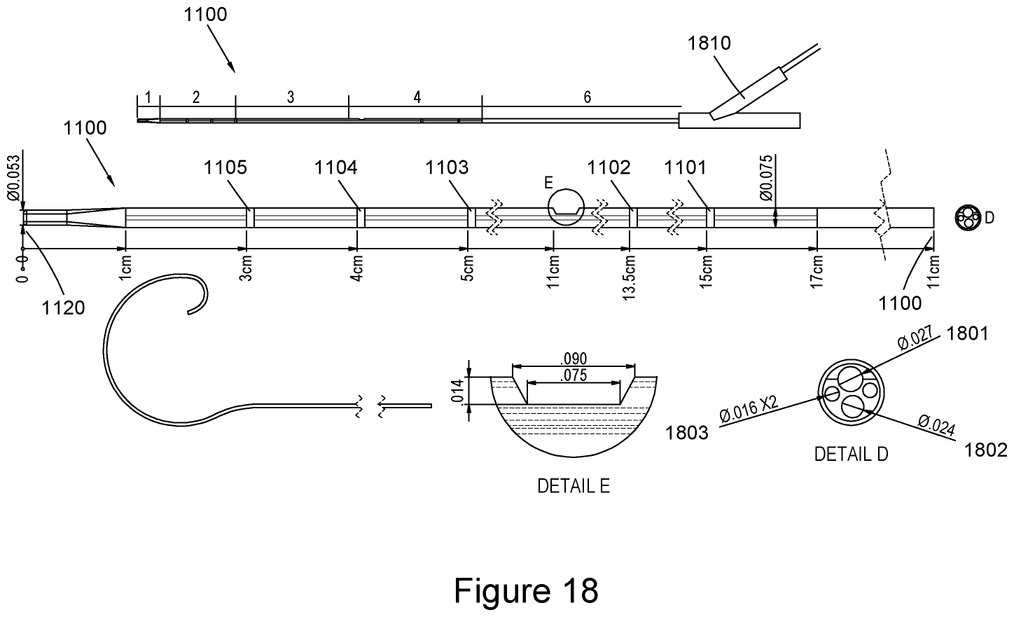

FIG. 18 shows further details of the catheter, shown in a pre-set configuration (i.e. straight, before curvature). As can be seen, at the proximate end 110 of the catheter 1100, there is provided a y-connector 1810, though which the pressure sensor wire 10 extends, as well as the electrode wire(s) 9. In the configuration where there are 5 electrodes, there may be provided 5 separate electrode wires. Each of the electrode wires may be welded to each marker band (i.e. the “electrodes” themselves).

The catheter may be provided with lumens 1801, 1802, and 1803. As can be seen, there may be two lumens 1803. The lumens maybe provided for any purpose. For example, lumen 1801 may have a diameter of 0.027 inches (0.069 cm), and may be provided for saline flush. Lumen 1802 may have a diameter of 0.024 inches (0.061 cm), and may be provided such that a stiff guidewire can be provided through it. The remaining lumens 1803 may have a diameter of 0.016 inches (0.041 cm), and be suitable for the electrode wire(s) 9 and pressure sensor wire 10 to pass through.

Also shown in FIG. 18 is area E, which may be suitable to house a pressure sensor. The area E may be a cutout on the surface of the catheter 110, having a depth of 0.014 inches (0.036 cm), and a width ranging between 0.09 inches (0.23 cm) at the surface, to 0.075 inches (0.191 cm) at the bottom. Whist in the example of FIG. 18, the cutout is positioned relatively near to proximate end, it would be appreciated that this could be placed closer to, or at the distal end to ease manufacturing, and therefore the pressure sensor be placed closer to the distal end.

At the proximal end 1110, the catheter may be coated with a braid wire 6, thereby providing a more robust catheter wire.

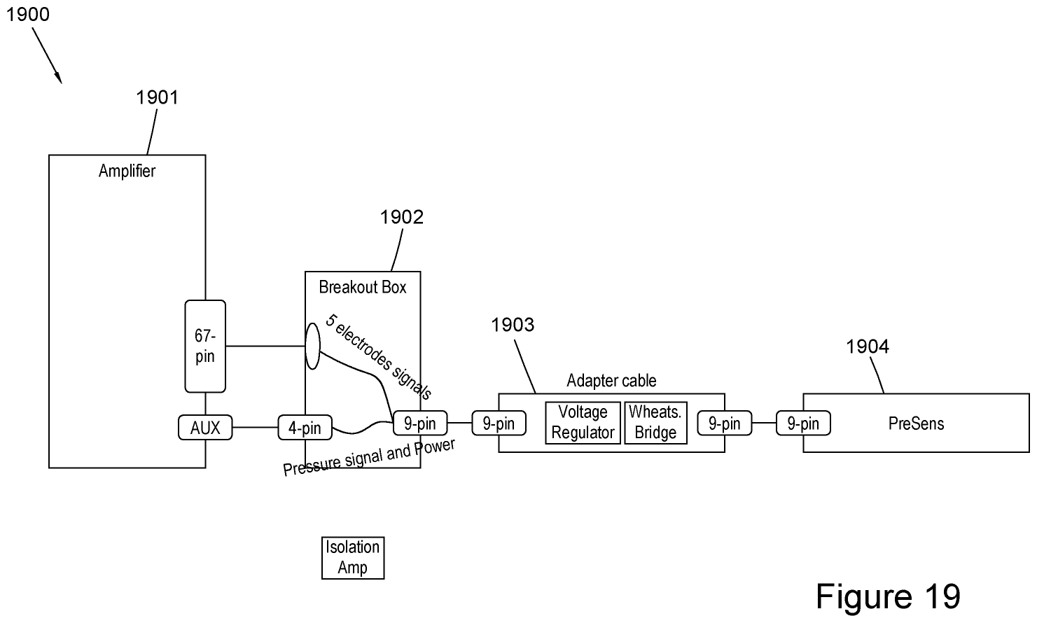

An example system 1900 that allows for the processing of signals received from a catheter described above is seen in FIG. 19, which may be used together with, or as an alternative to the system shown in FIG. 7. A catheter may connect to an amplifier 1901, amplifying the signals received by the catheter. A breakout box 1902 consolidates the electrode signals, the pressure signals and power to a single cable 1903, that can then be attached to a data processing module 1904, such as a PaCRTool data processing module, which may be configured to take the data measured by the various electrodes/sensors of the catheter 1100, and perform one or more of the methods discussed above.

1. A catheter for assessing cardiac function of a heart of a patient, the catheter comprising:
an elongate shaft extending from a proximal end to a distal end, the shaft having a first curve, a second curve, a third curve, and a fourth curve, the third curve having a diameter that is larger than that of the second curve, the shaft comprising:
at least one lumen for receiving at least one of a guidewire or a saline flush;
at least one first electrode disposed on the second curve of the shaft configured to, in use, contact a septal wall of a left ventricle of the heart, sense electrical signals, and apply pacing to the heart;
at least one second electrode disposed on the third curve of the shaft configured to, in use, contact a free wall of the left ventricle of the heart, sense electrical signals, and apply pacing to the heart;
at least one sensor disposed on the shaft for detecting an event relating to a rapid increase in a rate of pressure increase within the left ventricle of the heart a; and
communication means configured to transmit data received from the at least one first electrode, the at least one second electrode, and the at least one sensor.

2. The catheter of claim 1, wherein the at least one sensor is one or more of a pressure sensor, a piezoelectric sensor, a fiberoptic sensor, or an accelerometer.

3. The catheter of claim 1, wherein a stiffness of the shaft varies along a length of the elongate shaft between the proximal end and the distal end.

4. The catheter of claim 3, wherein a middle part of the shaft disposed between the proximal end and the distal end has a stiffness that is less than that of the proximal end, the shaft having a flexible tip at the distal end,
wherein the shaft comprises a multi-lumen extrusion having a first cover around the proximal end, a second cover around the middle part, and a third cover around the distal end, the first cover, the second cover, and the third cover having differing stiffnesses, and
wherein the multi-lumen extrusion comprises Pebax 63D, the first cover comprises Nylon 11 or Nylon 12 and a braid wire, the second cover comprises Pebax 35D, and the third cover comprises Pebax 75D or Pebax 55D.

5. The catheter of claim 1, wherein the at least one first electrode and the at least one second electrode are disposed along the shaft such that, in use, at least two electrodes of the at least one first electrode and the at least one second electrode are positioned opposing each other in the heart of the patient, and
wherein at least one electrode of the at least one first electrode or the at least one second electrode is configured to be placed in contact with a contralateral wall of the patient.

6. (canceled)

7. The catheter of claim 1, wherein the first curve is a counterclockwise curve having a first diameter, the second curve is a clockwise curve having a second diameter that is greater than the first diameter, the third curve is a clockwise curve having a third diameter that is greater than the first diameter and the second diameter.

8. The catheter of claim 7, wherein the first curve is an arc having an angle in a range between 33° and 45°, inclusive, the second curve is an arc having an angle in a range between 97.5° and 131.5°, inclusive, and the third curve is an arc having an angle in a range between 66.4° and 87.4°, inclusive, or
wherein the first diameter is in a range between 4.250 centimeters (cm.) and 5.750 cm., inclusive, the second diameter is in a range between 5.100 cm. and 6.900 cm., inclusive, and the third diameter is in a range between 1.275 cm. and 1.725 cm., inclusive.

9. (canceled)

10. The catheter of claim 1, wherein the shaft has a first thickness towards the proximal end, a second thickness that is less than the first thickness, and a third thickness at the distal end that is less than the second thickness,
wherein the first thickness is a diameter of 6 French (Fr), the second thickness is a diameter of 5 Fr, and the third thickness is a diameter of 3.5 Fr.

11. The catheter of claim 1, wherein the at least one first electrode comprises two first electrodes disposed on the second curve, and
wherein the at least one sensor is disposed on the second curve.

12. The catheter of claim 11, wherein the at least one second electrode comprises at least one second electrode or at least two second electrodes on the third curve and at least one second electrode on the fourth curve.

13. (canceled)

14. (canceled)

15. A system comprising:
the catheter of claim 1;
a signal amplifier;
a stimulator; and
a data processing module,
wherein the catheter is configured to be in signal communication with the stimulator, the signal amplifier, and the data processing module such that the at least one first electrode, the at least one second electrode, and the at least one sensor provide sensed data to the data processing module for further processing, and the at least one first electrode and the at least one second electrode provide pacing to the patient-s heart.

16. The system of claim 15, wherein the data processing module is configured to determine a characteristic response relating to an onset of myocardial synergy from the event relating to the rapid increase in the rate of pressure increase within the left ventricle of the heart.

17. The system of claim 16, wherein the at least one sensor is configured to provide pressure data regarding the pressure within the heart to the data processing module,
wherein the data processing module is configured to filter the pressure data to identify the characteristic response relating to the onset of myocardial synergy, and
wherein the characteristic response comprises a beginning of a pressure rise above a pressure floor in a pressure signal filtered above a first harmonic of the pressure signal.

18. The system of claim 17, wherein the characteristic response comprises a presence of high frequency components of the pressure signal or a band-pass filtered pressure trace crossing zero, and
wherein the high frequency components is above 40 Hz.

19. The system of claim 16, wherein the at least one sensor is configured to provide acceleration data from within the heart to the data processing module, and the data processing module is configured to filter the acceleration data to identify the characteristic response relating to the onset of myocardial synergy,
wherein the data processing module is further configured to calculate a center frequency of a continuous wavelet transform of the acceleration data to identify the characteristic response relating to the onset of myocardial synergy that is a peak of the center frequency, and
wherein the data processing module is further configured to average the center frequency over a number of heart cycles.

20. The system of claim 16, wherein the data processing module is configured to identify reversible cardiac dyssynchrony by identifying a shortening of a delay to onset of myocardial synergy as a result of pacing and by using the at least one sensor to measure a time of the event relating to the rapid increase in the rate of pressure increase within the left ventricle of the heart by identifying the characteristic response in the data received from the one or more sensors, the event relating to the rapid increase in the rate of pressure increase within the left ventricle being identifiable in each contraction of the heart, the data processing module being configured to measure the time of the event relating to the rapid increase in the rate of pressure increase within the left ventricle by:
processing signals from the at least one sensor to determine a first time delay between the measured time of the identified characteristic response relating to the rapid increase in the rate of pressure increase within the left ventricle and a first reference time;
comparing the first time delay with a duration of electrical activation of the heart to identify the presence of cardiac dyssynchrony in the patient when the first time delay is longer than a set fraction of electrical activation of the heart;
following pacing by one or both of the at least one first electrode or the at least one second electrode to the heart, calculating a second time delay between the identified characteristic response relating to the rapid increase in the rate of pressure increase within the left ventricle following pacing and a second reference time following pacing by:
measuring, via signals from the at least one sensor, the timing of the identified characteristic response; and
determining the second time delay and the second reference time following pacing;
comparing the first time delay and the second time delay; and
if the second time delay is shorter than the first time delay, identifying a shortening of a delay to onset of myocardial synergy (OoS) indicating that a time period until a point where all segments of the heart begin to actively or passively stiffen has shortened, thereby identifying the presence of reversible cardiac dyssynchrony in the patient.

21. The system of claim 20, wherein the data processing module is further configured to:
identify the absence of cardiac dyssynchrony in the patient if the first time delay is shorter than a set fraction of electrical activation of the heart; and
identify the absence of cardiac dyssynchrony in the patient if the first time delay is shorter than a set delay.

22. The system of claim 15, wherein the data processing module is configured to determine a degree of parallel activation of the heart undergoing pacing by:
calculating a vectorcardiogram (VCG) waveform or an electrocardiogram (ECG) waveform from a right ventricular pacing (RVp) and a left ventricular pacing (LVp);
generating a synthetic biventricular pacing (BIVP) waveform pacing by summing the VCG of the RVp and the LVp, or by summing the ECG of the RVp and the LVp;
calculating a corresponding ECG waveform or a VCG waveform from a real BIVP;
comparing the synthetic BIVP waveform and the real BIVP waveform; and
calculating time to fusion by determining the point in time in which the activation from the RVp and the LVp meets and a curve of the synthetic BIVP and a curve of the real BIVP start to deviate,
wherein a delay in time to fusion indicates that a larger amount of tissue is activated before wave fronts for electrical activation meet, thereby indicating a higher degree of parallel activation.

23. (canceled)

24. The system of claim 15 wherein the data processing module is configured to determine an optimal electrode number and an optimal electrode position for cardiac resynchronization therapy on the heart based on one or more nodes of a three-dimensional (3D) mesh of an at least part of the heart with a calculated degree of parallel activation of a myocardium above a predetermined threshold by:
generating the 3D mesh of the at least part of the heart from a 3D model of the at least part of the heart of the patient, or using a generic 3D model of the heart to obtain a 3D mesh of the at least a part of the heart, the 3D mesh of the at least a part of the heart comprising a plurality of nodes;
aligning the 3D mesh of the at least part of the heart to images of the heart of the patient;
placing additional nodes onto the 3D mesh corresponding to a location of the at least one first electrode and the at least one second electrode on the patient;
calculating a propagation velocity of an electrical activation between the nodes of the 3D mesh corresponding to the location of the at least one first electrode and the at least one second electrode;
extrapolating the propagation velocity to all of the nodes of the 3D mesh;
calculating the degree of parallel activation of the myocardium for each node of the 3D mesh; and
determining the optimal electrode number and the optimal electrode position on the heart of the patient based on the nodes of the 3D mesh with the calculated degree of parallel activation of the myocardium above the predetermined threshold.

25. The system of claim 15, wherein the catheter is configured to be provided into the heart through one or more of arterial access, venal access, subclavian access, radial access, or femoral access such that the at least one first electrode, the at least one second electrode, and the at least one sensor, in use, may be provided within the heart of the patient.
